(19) United States
(12) Patent Application Publication
(10) Pub. No.: US 2009/0166224 A1
(43) Pub. Date: Jul. 2, 2009

(54) Multi-lectin affinity chromatography and uses thereof

(76) Inventors: Ziping Yang, Livingston, NJ (US); William S. Hancock, Brookline, MA (US); Marina Hincapie, Framingham, MA (US)

Correspondence Address: WILMERHALE/BOSTON, 60 STATE STREET, BOSTON, MA, 02109, US

(21) Appl. No.: 11/579,610

(22) PCT Filed: May 5, 2005
(86) PCT No.: PCT/US05/15919

§ 371 Date: Feb. 11, 2009

Related U.S. Application Data
(60) Provisional application No. 60/568,466, filed on May 5, 2004.

(51) Int. Cl. G01N 27/26; C07K 1/22; C07K 14/00; C07K 1/00; G01N 33/68; G01N 33/566; C12Q 1/02; G01N 33/53; B01D 59/44

(52) U.S. Cl. 205/792; 530/413; 530/396; 435/272; 436/86; 436/501; 435/29; 435/7.92; 250/282

(57) ABSTRACT

Methods, compositions, and kits related to the use of multi-ligand affinity chromatography are described. The methods include those related to identification of glycoprotein panels for analyzing and diagnosing disease.

### CROSS-REFERENCE TO RELATED APPLICATIONS

This application claims priority to provisional U.S. Application Ser. No. 60/568,466, filed on May 5, 2004, which is herein incorporated by reference in its entirety.

### TECHNICAL FIELD

This invention relates to the field of disease diagnosis, glycoproteins, and more particularly to glycoprotein assays.

### BACKGROUND

Glycosylation plays a fundamental role in a diverse set of biological processes (Dwek et al., 2001, Int. J. Cancer 95:79-85) such as the immune response (Lowe et al., 2003, Arch. Dermatol. 139:617-621) and cellular regulation (Rudd et al., 2001, Science 291:2357-2364). Glycosylation can also be used as an indicator of environmental effects or cellular processes (Goochee et al., 1990, Biotechnology 8:421), is involved in signaling pathways associated with the transformation of a normal cell to a cancer cell (Alper, 2003, Science 301:159), and has been intimately associated with cancer. For example, glycosylation can affect tumor antigen interactions with receptors, e.g., CA125 with galectin (Seelenmeyer et al., 2003, J. Cell. Sci. 116:1305). Tumor cells show aberrant patterns of carbohydrates linked to cell surface proteins with the presence of larger, more branched N-linked oligosaccharides and the expression of certain glycoproteins has been associated with metastatic activity using experimental tumors (Gorelik et al., 2001, Cancer Metastasis Rev. 20:245-277; Gessner et al., 1993, Cancer Lett. 75:143-149).

The oligosaccharide structure of certain plasma proteins, e.g., transferrin and haptoglobulin, has been shown to change in disease or metabolic derangement (Peter et al., 1998, Biochim. Biophys. Acta 1380:93-101), and the rate of clearance of such proteins is largely controlled by the interaction of the asialo-form with the corresponding liver receptor. Accordingly, glycoproteins provide a useful source for detecting diseases such as cancer.

Biological fluids, including serum, can reflect disease in different organs and tissues with a change in secreted proteins, and as such, are commonly used as diagnostic fluids. In fact, glycoproteins are known to comprise a major part of the serum proteome (e.g., Anderson et al., 1998, Cancer Res. 19:1853-1861). However, there is a lack of global methods for the characterization of glycoproteins. Thus, what are needed are improved methods for assaying or profiling glycoprotein constituents in biological samples.

### SUMMARY

The invention relates to the development of methods to isolate glycoproteins, glycoconjugates, and the use of such glycoproteins to profile and diagnose disease.

Accordingly, in one aspect, the invention is a composition that includes at least three different ligands (e.g., lectins) attached to at least one solid support (e.g., at least four lectins or at least five lectins). In certain embodiments, the solid support includes at least one bead. In some cases, the solid support includes at least two beads and one type of lectin is attached to each bead. The solid support can include a gel (e.g., agarose). In some embodiments, the solid support is in a column; in other embodiments, the solid support is a microtiter plate or nanowell plate. Lectins in a composition can be concanavalin A (Con A), wheat germ agglutinin (WGA), Jacalin, lentil lectin (LCA), peanut lectin (PNA), Lens culinaris agglutinin (LCA), Griffonia (Bandeiraea) simplicifolia lectin II (GSLII) Aleuria aurantia Lectin (AAL), Hippeastrum hybrid lectin (HHL, AL), Sambucus nigra lectin (SNA, EBL), Maackia amurensis lectin II (MAL II), Ulex europaeus agglutinin I (UEA I), Lotus tetragonolobus lectin (LTL), Galanthus nivalis lectin (GNL), Euonymus europaeus lectin (EEL), or Ricinus communis agglutinin I (RCA). In certain embodiments, the lectins are present in equal ratios in the composition.

Another aspect of the invention relates to a method of isolating glycoproteins from a sample. The method includes contacting the composition as described above with a sample under conditions that promote binding of glycoproteins in the sample to the lectins, thereby providing a bound sample; removing an unbound sample from the contacted composition; and eluting the glycoproteins from the bound sample. In some embodiments of the method, the composition comprises at least three, four, or five different lectins. The sample can be, e.g., a biological fluid, a tissue preparation, of a cell culture preparation. A biological fluid can be, e.g., plasma, serum, blood, urine, lacrimal secretion, seminal fluid, vaginal secretion, sweat, saliva, or cerebrospinal fluid. In certain embodiments of the method, at least two different elution steps are performed.

Another aspect of the invention relates to a method of isolating a glycoprotein biomarker. The method comprises contacting a composition, comprising at least three different lectins attached to a solid support, with a sample containing the biomarker, under conditions that promote binding of glycoproteins in the sample to the lectins, thereby providing a bound sample, removing unbound sample from the contacted composition; and eluting at least one glycoprotein from the bound sample, wherein the biomarker is in the eluted sample. In some embodiments of the method, the eluted biomarker is isolated from the eluted glycoproteins. In some cases, the eluted biomarker is identified. The sample can contain, e.g., from 2 to 50 biomarkers. In some embodiments of the invention, the sample is a biological fluid, a tissue preparation, or a cell culture preparation. A biological fluid sample can be plasma, serum, blood, urine, lacrimal secretion, seminal fluid, vaginal secretion, sweat, saliva, or cerebrospinal fluid. In some embodiments, the method includes protease treating the eluted biomarker, e.g., with Asp-N protease, Glu-C protease, Lys-C protease, Arg-C protease, or trypsin. In other embodiments, the eluted biomarker is cleaved with a chemical, e.g., cyanogen bromide or hydroxylamine. In certain cases, the eluted biomarker is identified using mass spectroscopy. The unbound sample is, in some cases removed and can be used for analysis.

In another aspect, the invention relates to a method of detecting a change in the glycosylation of a biomarker. The method includes, contacting a composition as described above with a sample under conditions that promote binding of glycoproteins to the composition, thereby providing a bound sample; washing the composition to remove unbound components of the sample, thereby forming an unbound sample; eluting glycoproteins from the bound sample, thereby forming an eluted sample; detecting a selected biomarker in the unbound sample or in the eluted sample; and comparing the selected biomarker to a reference biomarker, such that a difference in the biomarker in the bound or unbound sample, relative to the reference biomarker indicates a change in glycosylation of the biomarker in the sample. In certain embodiments, the method includes quantitating the amount of the selected biomarker in the eluted sample or the unbound sample. The sample can be a biological sample, e.g., plasma, serum, blood, urine, lacrimal secretion, saliva, or cerebrospinal fluid. In some embodiments, at least one selected biomarker is detected in the unbound sample.

In certain aspects, the invention relates to a method of identifying a biomarker panel. The method includes contacting a sample comprising at least three different lectins attached to a solid support with a sample under conditions that promote binding of glycoproteins in the sample to the lectins, thereby providing a bound sample; removing unbound sample from the contacted composition; eluting glycoproteins from the bound sample, thereby providing a glycoprotein sample; identifying at least two proteins in the glycoprotein sample or the unbound sample, thereby providing a biomarker panel. The protein panel comprises at least three, four, five, ten, or fifteen proteins, e.g., glycoproteins. The sample can be from a subject having a disease or disorder. In some embodiments, the subject has or is at risk for having cancer, e.g., breast cancer. In certain embodiments, the method of identifying the glycoproteins includes the use of tandem mass spectroscopy (MS/MS), immunoassay, electrophoresis, normal phase HPLC with fluorescent detection, pulsed amperometric detection (PAD), a dye staining method, a fluorescent probe, surface plasmon resonance, MALDI-MS, MALDI-MS/MS, LC-MS/MS, LC-MS/MS, or LTQ-FTMS. In certain embodiments, the method of identifying the proteins includes an enzyme-linked immunosorbent assay (ELISA), dot blot, Western blot, two-dimensional gel electrophoresis, or capillary electrophoresis. The method can also include constructing a diagnostic glycoprotein panel by combining glycoprotein panels from at least two subjects having related conditions. In certain embodiments, the subjects have been diagnosed with or at risk for having a selected disease or disorder, e.g., cancer (for example, breast cancer). In other embodiments, the subject has or is at risk for having cardiovascular disease. In certain embodiments, a glycan, e.g., a mucin or a glycosoaminoglycan, is captured.

In some aspects, the invention relates to a method of diagnosing the presence of a disease or disorder in a subject. The method includes contacting a composition as described above with a sample from a subject under conditions that promote binding of glycoproteins in the sample to the lectins, thereby providing a bound sample; removing an unbound sample from the contacted composition; eluting the glycoproteins from the bound sample; identifying the presence of at least two glycoprotein biomarkers in the sample, such that the presence of the biomarkers indicates the presence of a disease or disorder in the subject.

Another aspect of the invention relates to a method of identifying a subject at risk for a disease or disorder. The method includes contacting a composition as described above with a sample from subject suspected of being at risk for a disease of disorder under conditions that promote binding of glycoproteins in the sample to the lectins, thereby providing a bound sample; removing an unbound sample from the contacted composition; eluting the glycoproteins from the bound sample; and analyzing the sample for at least two glycoprotein biomarkers that can indicate that a subject is at risk for a disease or disorder.

Another aspect of the invention relates to a composition that includes at least three different affinity ligands attached to at least one solid support. In some embodiments, at least one of the affinity ligands is an IMAC ligand, heparin, a histone, calmodulin, or a lectin.

Other features and advantages of the invention will be apparent from the detailed description, drawings, and from the claims.

### DESCRIPTION OF THE DRAWINGS

FIG. 1 is a photographic reproduction of a one-dimensional gel of proteins isolated using M-LAC and Schiff's base stained. Lane 1: Horseradish peroxidase (positive control), lane 2: Flow-through, lane 3: First elution sample, lane 4: Elution sample from a replicate experiment, lane 5: Jacalin-selected proteins captured by the multi-lectin column, lane 6: Con A-selected proteins, lane 7: WGA selected proteins, lane 8: Molecular weight standards (negative control).

FIG. 2 is a representation of LTQ MS spectra for three proteins.

FIG. 3 is a graphic representation illustrating the results of binding assays of IgY antibodies that bind to α-1-antitrypsin-coated plates using an ELISA format.

FIG. 4 is a graphic representation illustrating the results of lectin-ELISA to detect differential binding of four different glycoprotein standards to the lectin array.

FIG. 5 is a graphic representation of an MS/MS spectrum for the Her2 peptide, ¹⁴⁴SLTEILKGGVLIQR¹⁵⁷ (SEQ ID NO:1). Charge state: +3; Xcorr: 3.04; DetaCn: 0.12; Rsp:1

FIG. 6 is a diagrammatic representation of the fragmentation of the peptide SLTEILKGGVLIQR (SEQ ID NO:1).

FIG. 7 is a graphic representation of data collected in a full MS scan at the retention time where the peptide ion of SLTEILKGGVLIQR (SEQ ID NO:1) of ERB2 was identified.

FIG. 8A is graphic representation of an extracted ion chromatogram of the peptide of ceruloplasmin, DLYSGLIGPLIVCR (SEQ ID NO:2) (M/Z signal 788.6) from M-LAC enriched breast cancer patient serum samples.

FIG. 8B is graphic representation of an extracted ion chromatogram of the peptide of ceruloplasmin, DLYSGLIGPLIVCR (SEQ ID NO:2) (M/Z signal 788.6) from M-LAC enriched breast cancer patient serum samples.

FIG. 8C is graphic representation of an extracted ion chromatogram of the peptide of ceruloplasmin, DLYSGLIGPLIVCR (SEQ ID NO:2) (M/Z signal 788.6) from M-LAC enriched breast cancer patient serum samples.

FIG. 8D is graphic representation of an extracted ion chromatogram of the peptide of ceruloplasmin, DLYSGLIGPLIVCR (SEQ ID NO:2) (M/Z signal 788.6) from M-LAC enriched breast cancer patient serum samples.

FIG. 8E is graphic representation of an extracted ion chromatogram of the peptide of ceruloplasmin, DLYSGLIGPLIVCR (SEQ ID NO:2) (M/Z signal 788.6) from M-LAC enriched breast cancer patient serum samples.

FIG. 9 is a photographic representation of an IEF gel separation profile of transferrin, asialotransferrin, and their fractions collected from M-LAC. Lanes 3 and 7: unbound proteins from flow-through; lanes 4, 5, and 6: fractions from transferrin displacement, and lanes 8, 9, and 10: fractions from asialotransferrin displacement

FIG. 10A is a graphic representation of an extracted ion chromatogram of the transferrin peptide, “KPVEETANCHLAP” (M/Z signal 530.2), for M-LAC Con A displacer fraction from a serum sample.

FIG. 10B is a graphic representation of an extracted ion chromatogram of the transferrin peptide, “KPVEETANCHLAP” (M/Z signal 530.2), for M-LAC Con A displacer fraction a neuraminidase-treated serum sample.

FIG. 10C is a graphic representation of an extracted ion chromatogram of the transferrin peptide, “KPVEETANCHLAP” (M/Z signal 530.2), for M-LAC WGA displacer fraction from a serum sample.

FIG. 10D is a graphic representation of an extracted ion chromatogram of the transferrin peptide, “KPVEETANCHLAP” (M/Z signal 530.2), for M-LAC WGA displacer fractions from a neuraminidase-treated serum sample. Within the acceptable retention time window, a small peak area of 1.6 was integrated from the WGA fraction of this sample.

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Methods, compositions, and kits are described herein that utilize Multi-Lectin Affinity Chromatography (M-LAC) for the assay of glycoproteins.

The implementation of a global approach to glycoproteomics requires the development of a simple-to-use relatively high throughput method for screening complex biological samples for changes in glycosylation. A major challenge is the detection of so-called ‘silent changes’ in protein glycosylation that can occur (e.g., disease or changes due to normal changes in cellular physiology), but that are not associated with an identified change in protein function. Identification of silent changes and correlation of these changes with changes to cells permits monitoring subtle changes in glycosylation patterns, e.g., in the absence of a change in protein expression level. M-LAC provides a method of enriching for glycoproteins and therefore can be used in methods that involve detection of silent changes in glycosylation.

M-LAC involves contacting a sample with multiple glycoprotein ligands (e.g., lectins) that may be attached to a substrate to form an affinity support. The components of the sample that do not bind to the ligands are removed (e.g., by washing), and in some cases, are collected for analysis. The sample components that bind to the molecules of the affinity support (e.g., glycoproteins or other glycoconjugates) are, in some embodiments of the invention, recovered. Recovery of bound glycoproteins is generally accomplished using displacer molecules, such as, but not limited to, sugars that specifically bind to the ligands. The recovered (i.e., isolated) glycoproteins can then be used for further analyses, e.g., to determine the types glycoproteins present in the sample, the abundance of glycoproteins in the sample, the types of oligosaccharides associated with the glycoproteins, or the amounts of oligosaccharides associated with the glycoproteins. Such analyses are accomplished using methods known in the art such as LC-MS/MS. In some embodiments of the invention, unbound molecules (e.g., non-glycosylated proteins) are recovered and analyzed as described herein. “Unbound molecules” refers to proteins that are not captured on a particular M-LAC column and are generally captured in a flow-through fraction when loading an M-LAC column or other M-LAC format. Therefore, the proteins in an “unbound” or “flow-through” sample or fraction may contain glycoproteins having oligosaccharide moieties that do not bind to the molecules on a specific column. In general, an unbound sample is composed primarily of non-glycosylated proteins.

In some embodiments of the methods of the invention, a glycoprotein sample is compared to a reference. The reference can be a control. A reference can be a previously established number or set of numbers suitable for comparison or it can be a control. For example, a sample from a subject having a disease can be compared to a sample from a control subject that does not have the disease. Glycoproteins that are different in type or abundance between the two samples can be used as biomarkers of disease, e.g., for diagnostic purposes, and are candidates for drug targets. Such biomarkers can also be used to monitor the response of a subject to a therapy, e.g., drug therapy.

M-LAC can be coupled upstream or downstream of other chromatographic separation methods, analytical technologies, or diagnostic technologies or platforms. For example M-LAC can be combined with immunoaffinity depletion strategies that are used to remove abundant proteins from a sample before performing M-LAC. Non-limiting examples of such proteins that it can be desirable to remove include albumin, α and β immunoglobulins, transferring, haptoglobin, and fibrinogen. Flow-through and bound fractions recovered from M-LAC can be further fractionated by other separation methods such as, e.g., chromatography or electrophoresis as described herein.

M-LAC is useful for a number of different applications. For example, glycoprotein and non-glycoprotein protein profiling; relative measurements in abundance of differentially expressed proteins; detecting changes in glycoprotein patterns; detecting interindividual glycoprotein expression or inactivation; development of diagnostics; theranostics (the use of diagnostic testing to diagnose a disease, characterize a disease, select a treatment regime, and monitor patient response to therapy); identification of biomarkers for drug discovery and development; therapeutic glycoprotein development (e.g., to monitor process changes, process qualification/validation, or lots used in clinical trials); monitoring fermentation processes; assaying cell culture changes (e.g., in growth and high cell density cultivation for optimizing biomass yields and growth rate), or in the purification of a glycoprotein therapeutic. A biomarker can be a single marker or a glycoprotein profile or glycoprotein pattern change.

The present invention ameliorates problems associated with some proteomic strategies and designs in which low abundance proteins are under-represented. M-LAC provides an enrichment method that can concentrate low abundance glycoproteins, e.g., at least two-fold, from a complex sample. M-LAC can be integrated with protocols for detecting modulation (up-regulation or down-regulation) of glycosylated and non-glycosylated proteins from the same sample. M-LAC can also be used for monitoring changes in glycosylation that occur due to a physiological perturbation of a biological system.

In general, M-LAC methods assay changes between a control sample(s) and a test sample, e.g., a perturbed sample, e.g., from a subject at risk for or having a disease or disorder. Perturbation refers to any change in state related to, e.g., disease, treatment, or different cell growth conditions.

Useful glycoprotein ligands are lectins, but the invention is not limited to lectins. Other ligands with biological affinities can be used in a similar format to isolate proteins having specified features in common. This approach known as Multi-Affinity Protein Separation (MAPS) enables separation of proteins based on fractionation of proteins into different protein families or classes such as charge (positive or negative). As in M-LAC, MAPS involves the use of multiple molecules (e.g., two, three, four, or five) that bind to a specified class of proteins. The molecules used in a MAPS composition can each bind to proteins based on different features, e.g., one binding molecule used in a MAPS can be a lectin and a second can be, e.g., heparin, a histone, or calmodulin. An advantage of MAPS is the ability to target specific classes of proteins, e.g., when coupled to M-LAC to further fractionate a flow-through or bound fraction to decrease the complexity of a sample and thus be able to increase protein coverage and better dynamic range.

At least 160 lectins are known and can be obtained. Representative lectins include, without limitation, Concanavalin A (Con A), wheat germ agglutinin (WGA), Jacalin lectin (Jacalin), Aleuria aurantia lectin (AAL), Hippeastrum hybrid lectin (HHL, AL), Sambucus nigra lectin (SNA, EBL), Maackia amurensis lectin II (MAL II), Ulex europaeus Agglutinin I (UEA I), Lotus tetragonolobus lectin (LTL), and Galanthus nivalis lectin (GNL). In some embodiments of the invention, sialic-specific M-LAC compositions can be made by mixing LTA, SAB, and MALII in equal ratios. To select galactose-specific glycoproteins a combination of Jacalin, Euonymus europaeus lectin (EEL), and Ricinus communis agglutinin I (RCA). Commercial sources of lectins include Vector Laboratories, Inc. (Burlingame, Calif.), GALAB Technologies (Geesthacht, Germany), and Sigma (St. Louis, Mo.). Alternatively, lectins can be isolated from natural sources or synthesized.

The specificity and affinity of lectin binding in biological systems are significantly increased by a membrane clustering effect. Accordingly, polymeric displacers can also be used to elute glycoproteins from affinity support used in M-LAC. Additionally, polymeric displacers can also be used to determine the structure-function relationships of given displacers, and for generating various glycoprotein fractions. Examples of such polymeric displacers include, without limitation, Man Cα(1,6){Man Cα(1,3)Man}5SMan Cα(1,6){Man Cα(1,3)Man} (meso-tetra(sulfonatophenyl) porphyrin, meso-zinc-tetra(sulfonatophenyl) porphyrin (K carboxylmethyldextran (CMD) (Me α-D-mannopyranoside, D-mannopyranoside, Me α-D-glucopyranoside, D-glucopyranoside (p-nitrophenyl 1-thio-α-mannoside, 4-methylumbelliferyl α-D-mannopyranoside, ConA-La³⁺-α-methylmannopyranoside, myo-inositol, and methyl α-D-GalNAc (Sigma-Aldrich, St. Louis, Mo.). Polymeric displacers are used under conditions consistent with displacement chromatography (Frenz et al., 1985), or modifications such as elution-modified displacement chromatography (Wilkins et al., 2002) and sample displacement chromatography (Torres and Peterson, 1990, and Manseth et al., 2004).

The invention also relates to methods of identifying lectins suitable for use in M-LAC of specified sample types such as tissue samples, cell culture sampled, or samples derived from specific body fluids. High throughput assays can be used to identify suitable lectins. For example, a lectin can be bound to a multi-well plate (Thompson et al., 1989, Clin. Chim. Acta 180(3):277-284), and the ability of the lectin to bind a desired fraction or glycoprotein(s) is determined using, e.g., an ELISA assay. A lectin that binds a desired sample is suitable for use in an M-LAC procedure to isolate glycoprotein from a sample containing the glycoprotein. In another approach, a multiwell plate is coated a glycoprotein or glycoprotein sample, incubated with a panel of labeled (e.g., biotinylated lectins) and the amount of each lectin bound to the glycoprotein sample is assayed, for example, using an avidin-alkaline phosphatase conjugate (Duk et al., 1994, Anal. Biochem. 221(2):266-272 and Goodarzi and Turner, 1997, Glycoconj. J. 14(4):493-496).

The methods of the invention include additional applications of high-throughput methods. In a non-limiting example, combinations of ligands (e.g., lectins) are bound to nanowell plates. The plate is contacted with a sample, washed (the unbound fraction can be collected), and the bound fractions eluted using specific displacers. A large array (e.g., tens, hundreds, or thousands) of combinations can be tested in this manner. Such applications are useful, e.g., for identifying changes in glycosylation patterns in a set of samples.

Derivatized lectins can also be useful in the method of the invention, e.g., for attaching lectins to a support. For example, biotin-tagged lectins are either purchased from suppliers (e.g., Vector Laboratories, Burlingame, Calif.) or tagged with a N-hydroxysuccinimide derivative of biotin after protecting the active site of the lectin with a monosaccharide (see, e.g., Berger et al., 1994). The biotin-labeled lectins are then bound to a 96 well plate coated with, e.g., avidin (Thompson et al, 1989, Clin. Chim. Acta 180(3):277-84) for use in an ELISA format for screening (see Examples, infra). Other labels that can be used include, without limitation. Other useful labels include, without limitation, fluorescent probes such as rhodamine or FITC, radioactive labels, electroactive labels, affinity tags that can conjugate with secondary labels, oligonucleotides for PCR amplification, and chromogenic peptides.

As with M-LAC, any other mixed natural or synthetic ligands can be used in combination to provide a multiple-affinity format (MAPS). Molecules that can be used in this format include, but are not limited to heparin, protein receptors, adhesion molecules, glycosoaminoglycans, or other biologically active compounds such as calmodulin, ATP, enzyme inhibitors or substrates ligands that mimic the specificity and dynamic co-operativity achieved by multivalent interactions at the surface of cells. The interaction of multiple ligands results in increased specificity, higher binding affinity and better capture of mixtures of proteins in complex samples. For example, MAPS can provide a useful method for isolation of glycoproteins, kinases, Ca²⁺ binding proteins, or growth factors. Unlike a single ligand column format, the combination of two or more ligands into a single column (MAPS or M-LAC) can mimic in vivo recognition systems. For example, the M-LAC approach can be viewed as a multi-receptor complex with multiple glycans recognition domains. A single lectin binding to a single saccharide ligand typically has a low affinity and weak interaction, with Kd values on the order of micromolar to millimolar ranges. To overcome these weak interactions and to accomplish the diverse biological functions the different lectins and the carbohydrate ligands take advantage of affinity enhancements due to multiple interactions at the surface of the cell.

In a single column approach such as M-LAC, glycoproteins are captured solely based on the normal “on-off” mechanism of affinity chromatography. Therefore, the efficiency of ligand capture depends on a high binding constant. Unlike a single lectin column, the low affinity of lectins for their glycoproteins is overcome by combining several lectins into a single column and thus increasing the multivalent interactions which results in increase effective affinity an therefore better yield and specific capture of plasma/serum, tissue lysates and cell culture derived glycoproteins. Alternatively, the M-LAC or other mixture of ligands used in MAPS can be used to fractionate a complex mixture into two or more distinctly glycosylated fractions (as in M-LAC) or into different family-specific protein fractions (as in MAPS) by eluting with a series of different displacers specific for each ligand in the column, thus achieving a fractionation of the different biological motifs. For example, in M-LAC, the approach is a powerful method to detect different glycosylation states of biomarkers in disease by the shift of a glycoprotein form one displacer fraction to another. A key innovation of the M-LAC concept is the observation that the distribution of glycoproteins in each lectin displacer fraction is determined by the structure of the glycoproteins. Therefore, the distribution of a given protein in the different displacer fractions will reflect shifts in the glycosylation pattern. For example, after determining the distribution of a glycoprotein using a three ligand column (e.g., an M-LAC column loaded with a composition containing three different lectins), three displacer fractions of an appropriate normal sample can be compared to a disease sample to identify any shift in the fraction in which a protein is located. Such methods can be used to determine whether a putative disease biomarker has a change in the type or degree of glycosylation.

In a MAPS format, a combination of affinity ligands are present in a composition. Examples of affinity ligands include but are not limited to calmodulin to isolate Ca²⁺ binding proteins, molecules used in immobilized metal affinity chromatography (IMAC) to isolate metal binding proteins, heparin to isolate growth factors, lipoproteins, or proteins with a lysine-rich domain, lysine-affinity for isolation of plasminogen, plasminogen activator or other nucleic acid binding proteins, 5′ AMP for ATP-kinases, and protease inhibitors (e.g., benzamidine) for isolation of proteases. M-LAC and MAPS can be used separately or in combination for the global and/or targeting profiling of proteins from any biological sample. For example a mixture of calmodulin resin and benzamidine can be used to separate two different classes of proteins, Ca²⁺-specific proteins and serine proteases.

The invention provides, in part, a multi-lectin affinity composition for use in the method of the invention. This composition includes at least two ligands, such as two lectins, bound to a support, thus providing an affinity support. Examples of such supports include, without limitation, a bead or microbead composed of silica, agarose, or a polymer, a plate (e.g., a microtiter plate), slide (e.g., glass or polymer slide), nanowell plate, or polyethylene glycol or other soluble polymer that can be precipitated or isolated by some other physical process to which a lectin is bound. The lectins used in the invention can be attached to the support directly or indirectly (e.g., using an antibody or biotin) using methods known in the art, e.g., using aldehyde functionalized resins or linkers such as cyanogen bromide, carbonyl diimidazole glutaraldehyde, epoxy, periodate, or bisoxirane (Harris et al, 1989, In Protein Purification Methods. A Practical Approach, IRL Press, New York, N.Y.). In the case of particulate supports such as agarose beads, a mixture of lectins may be attached to a single bead, or in certain embodiments, a single type of lectin is attached to each bead and the mixture of lectins used in the composition is made by mixing at least two of these types of beads. Alternatively the lectin may be attached to a restricted access media for the purposes of selecting glycosylated molecules of different molecular weight ranges.

Compositions according to the invention can also be constructed using commercially available components such as agarose-bound lectins (see, e.g., Vector Laboratories, Burlingame, Calif.).

The particular lectins selected for use in an M-LAC composition are generally determined based on the type of glycoprotein targeted for capture. In some cases, the lectins are selected based on empirical determinations, e.g., using high throughput procedures as described infra. In other cases, an M-LAC composition includes lectins are selected based on their ability to bind to a specific glycosylation structure, such as fucose, sialic acid, galactose, or any other specific sugar. For example, for global analysis (e.g., of plasma proteins), the M-LAC composition can contain a mixture of Con A, WGA, and Jacalin. In general, the lectins in an M-LAC composition are present in approximately equal amounts. Additional examples of M-LAC compositions are those that include Aleuria aurantia lectin (AAL), Hippeastrum hybrid lectin (HHL, AL), Sambucus nigra lectin (SNA, EBL), Maackia amurensis lectin II (MAL II), Ulex europaeus Agglutinin I (UEA I), Lotus tetragonolobus lectin (LTL), and Galanthus nivalis lectin (GNL). In some embodiments of the invention, sialic-specific M-LAC compositions can be made by mixing LTA, SAB, and MALII in equal ratios. To select galactose-specific glycoproteins a combination of Jacalin, Euonymus europaeus lectin (EEL), and Ricinus communis agglutinin I (RCA) can be utilized. The last two types of M-LAC compositions (LTA/SBA/MALII and Jacalin/EEL/RCA) are particularly useful for monitoring glycosylation changes associated with cancer and other diseases (Kawado et al. 2004, Arch. Biociem. Biophys. 426(2):306-313; Sillanaukee et al., 1999, Eur. J. Clin. Invest. 29(5):413-425).

The combination of lectins used in a specific M-LAC system can be selected based on the results of specific displacement studies. For example, studies (described in the Examples infra) lead to the selection of Con A, WGA, and Jacalin lectin for M-LAC of plasma or serum glycoproteins.

M-LAC can be optimized for a particular sample type or to achieve particular results. For example, various combinations of lectins can be tested for their ability to be used in M-LAC to enrich for glycoproteins in general, or for particular glycoproteins in a sample. Lectin density can also be tested to achieve an optimal recovery of glycoproteins. Other variables known in the art such as temperature, flow rate, and parameters related to the sample application can also be tested to identify those conditions best suited to a particular M-LAC application.

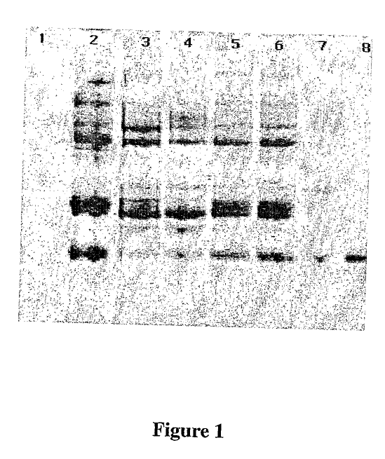

The characteristics of a set of M-LAC conditions can be readily analyzed, e.g., by analyzing the M-LAC products (i.e., glycoprotein and/or non-glycoprotein fractions). For example, one-dimensional gels can be used to analyze the contributions of each lectin in an M-LAC run under specified conditions. FIG. 1 illustrates such an analysis in which serum glycoproteins were isolated using an M-LAC protocol in which a column containing agarose-bound concanavalin A (Con A), wheat germ agglutinin (WGA), and Jacalin were the lectins. In these experiments, M-LAC was carried out at neutral pH and glycoproteins eluted from the column in three fractions, each by displacement using a specific monosaccharide and then analyzed using one-dimensional gel electrophoresis. Note that each lectin fraction contains a different distribution of glycoproteins, illustrating the use of M-LAC for fractionating differently-lectin binding glycoproteins by a method that permits a single sample loading followed by sequential elution, e.g., without having to load and elute each fraction separately.

For certain embodiments of the invention, for example, for diagnostic uses of M-LAC, it is desirable to determine the panel of lectins that can be used to isolate glycoproteins of interest for a particular application. In certain applications, a database of clinically significant glycoproteins is established based on serum, plasma, tissue biopsy, cell culture studies, or a combination thereof, which provide information on, for example, the types of glycosylation, source of the material, commercial availability and disease specificity. Such informative glycoproteins are referred to herein as “standards.” A subset of these standards (about 5 to 10) are used to develop an M-LAC suitable for identifying the selected standards in a test sample, i.e., an M-LAC that will recover the standards, when present, in a test sample. A specific glycoprotein is selected, if available, in a sample batch large enough for the planned study. In general, about 0.5 mg to 5 mg (e.g., 1 mg to 2 mg) is the amount of a standard used in establishing an M-LAC procedure. This amount can be adjusted, e.g., based on the sensitivity of detection assays used to quantitate the amount of glycoprotein present to assure detection of the standard. A non-limiting example of a useful standard is transferrin.

In certain embodiments, IgY antibodies (e.g., from GenWay Biotech, Inc., San Diego, Calif.) that specifically bind to selected glycoprotein standards are used. The specificity of the antibody to the protein portion of the glycoprotein is tested by removal of the carbohydrate with N-glycanase or cleavage of O-linked sugars using methods known in the art.

In general, at least two, e.g., three, four, or five lectins mixed in a predetermined ratio. In general, the lectins are present in a composition in approximately equal amounts (e.g., 2:1:1 (ConA:WGA:Jacalin), 1:1 (Con A:Jacalin), and 1:1:1 (LTL:MAL II:SNA).

The compositions used for M-LAC can be scaled up or miniaturized depending on the sample volume. The compositions can include a column (e.g., packed with an M-LAC affinity support) that is designed for collection of a sample using, e.g., HPLC or gravity.

In general, M-LAC compositions are prepared by attaching (e.g., covalently attaching) at least two (e.g., at least three, at least four, or at least five) lectins to a solid substrate or to a molecule that is attached to a solid substrate. In certain embodiments of the invention, commercially available linkage products can be used to prepare a composition. For example, commercially available agarose-bound lectins can be combined to produce a composition (see, e.g., Examples).

In some embodiments of the invention, a M-LAC composition includes a multi-lectin column that is prepared by physically mixing at least two (e.g., three, four, or five) lectins that are immobilized on a substrate that provides a solid support such as a gel (e.g., agarose, silica, or polymeric resins). The amount of a specific immobilized lectin that is used in a method of the invention is based on the sample concentration and approximate level of a specified glycoprotein motif (i.e., a carbohydrate motif that can bind to the specific, immobilized lectin). In general, the amount of the lectin is present in the composition to be contacted by a sample in excess of at least about 50% (e.g., at least 75% or at least 100%) over the amount of the portion of the sample predicted to bind to the lectin. Alternatively, lectins are immobilized on a solid support at various lectin/solid support ratios or concentrations. The binding capacity of the lectin/solid support composition is determined, or the amount of a sample that can be loaded without saturating the column is determined. In general, it is desirable that an amount of lectin be in a composition that is in at least two-fold excess of the amount of molecule that is to be bound (e.g., a ten-fold excess or a 100-fold excess). A solid support such as agarose is used in a gravity flow column format or with only mild pressure to avoid undesirable compression of the support. Non-limiting examples of M-LAC affinity compositions according to the invention include those containing at least two of Con A, WGA, Jacalin, LTL, MAL II, or SNA bound to a solid support (e.g., agarose).

The M-LAC method of the invention can be performed using a batch method, spin column, or magnetic bead format. Automated high throughput formats can be used, e.g., using a vacuum manifold, HPLC, or robotic format. In some embodiments of the invention, a sample is fractionated using M-LAC having a particular composition and a fraction collected is re-fractionated using a second M-LAC having a particular composition that is different from the first. For example, a fraction from the first M-LAC (e.g., Con A/WGA/Jacalin) column can be further fractionated on a second M-LAC column (e.g., containing LTA, MAL II, and SAB) to select only for those glycoproteins containing sialic acid. Alternatively, a fraction collected from a first column (e.g., ConA/WGA/Jacalin M-LAC) can be can loaded onto a second M-LAC column, e.g., containing Lens culinaris agglutinin (LCA) and Griffonia (Bandeiraea) simplicifolia lectin II (GSLII)) to specifically isolate α-linked mannose and N-acetylglucosamine-containing glycoproteins.

The samples analyzed by the method include body fluids, tissues, and cell cultures. Body fluids include, without limitation, plasma, milk, serum, blood, saliva, urine, nipple aspirate, sweat, tumor exudates, joint fluid (e.g. synovial fluid), inflammation fluid, perspiration, lacrimal secretions, semen, and vaginal secretions. Tissue derived samples can be from any part of the body, and can be from cell organelle preparations, cytoplasm, membrane, or nuclei. Cell cultures can be from an animal, mammal, e.g., a non-human mammal such as a mouse, rat, goat, pig, sheep, horse, or cow), human, fungus or bacterium. Samples can be prepared before subjecting to M-LAC, e.g., using methods known in the art that solubilize proteins or dissociate proteins from other proteins and cellular components.

In some embodiments of the invention, a sample, such as a cell lysate, is treated with one or more protease inhibitors to minimize proteolysis (e.g., phenylmethanesulfonyl fluoride (PMSF), aprotinin, chymostatin, EDTA, pepstatin A, or leupeptin). Detergents such as 1% Triton® X-100 can also be used to facilitate recovery of proteins and to minimize proteolysis. Other examples of detergents that can be used include, without limitation, Tween 20, Tween 80, and octyl glucoside. In some embodiments of the invention, a zwitterionic detergent is used such as, without limitation, Zwittergent 3-14, 3-12, 3-10, CHAPS, deoxycholate, NP40, or any other non-denaturing detergent. Supplements such as protease inhibitors and detergents are typically included in loading buffer and, optionally, are included in elution buffers.

Sample preparation can be adjusted to increase recovery of, e.g., glycoproteins. For example, after isolating cells from a sample such as a tissue or cell culture, the cells can be lysed with different buffers to identify conditions that result in a useful amount and quality of recovery of the cellular proteins. Typical lysis conditions are 4° C. for 30 minutes in a buffer that contains suitable enzyme inhibitors such as NaF, a Sigma protease inhibitor cocktail (Sigma-Aldrich, St. Louis, Mo.) and detergents such as CHAPS, Na deoxycholate, Triton® X-100, or a combination thereof. The detergent concentrations in the lysis buffer are generally about 0.25% to about 2.0%, e.g., about 0.5% to about 1.0%. Other enzyme inhibitors and detergents are known in the art and can be tested for their ability to produce a suitable sample. In some embodiments of the invention, a sample is prepared using sonication. Manual homogenization is used in some preparation protocols, e.g., using a tissue homogenizer such as a ground glass homogenizer, Potter-Elvehjem homogenizer, or other Teflon-coated pestle homogenizer.

A cell or tissue sample can also be prepared using the techniques described above combined with methods that fractionate cell components, e.g., that separate membrane and cytosol. Such methods are known in the art and include differential centrifugation.

The loading and elution buffers used in the protocols in which gel (e.g., agarose) supports are used are generally mild, i.e., the buffers used are generally at a physiological pH, (e.g., using Tris at pH 7.4), do not contain chaotropes such as 8 M urea or 6 M guanidine since these can result in disruption of the three dimensional structure or stripping of the lectins, and no reducing agents are used such as dithiothreitol (DTT). For example, a loading buffer can be Tris buffer, pH 7.4 containing 0.15 M NaCl, Hepes buffer, pH 7.4, or PBS.

Glycoproteins bound to the lectins in an M-LAC procedure are generally eluted from the lectins using an elution buffer containing molecules that compete for binding to the lectin, e.g., molecules including a sugar moiety that can bind to a lectin used in the M-LAC such as a monosaccharide that binds to a specific lectin. Typically, the displacing molecules are saccharides such as methyl-α-D-mannopyranoside, N-acetyl-glucosamine, and galactose. In general, the elution buffer is the same as the loading buffer with the addition of a higher concentration of salt, typically about 0.5 M NaCl and the displacing molecules. Displacing molecules are generally present in a concentration of, e.g., from about 0.1 M to about 1 M, although it is also possible to empirically determine concentrations of displacing molecules for use in a method.

One advantage of using M-LAC compositions in which the solid support such as agarose, is that the conditions for loading and elution of glycoproteins are typically mild, and this results in, e.g., relatively long column lifetimes. For example, a column can be used for multiple cycles of sample fractionation, e.g., at least 10 cycles, at least 25 cycles, at least 50 cycles, or at least 100 cycles. Such columns can also maintain good recovery of active proteins over multiple cycles, e.g., recovery of at least 75% of the proteins, at least 80% of the proteins, at least 90% of the proteins, or at least 95% of the proteins. Columns can be regenerated, e.g., by washing with a high salt buffer and, optionally, a denaturating agent such as 2 M urea.

In a typical M-LAC using an agarose composition, the loading, washing, and separation steps are performed at 4° C. In some cases, the columns are not regenerated to avoid carryover between different sample analyses.

While agarose does not tolerate high pressures and is therefore not amenable to, e.g., an HPLC format, such compositions are compatible with a wide range of biological samples and, in general, gives good recovery of biopolymers, and so are useful for certain applications.

In certain embodiments of the invention, an M-LAC fraction (either the wash fraction (unbound fraction) or the eluted fraction (glycoprotein fraction)) is trypsinized prior to analysis or can be further fractionated by other chromatographic methods known in the art, including those described herein. The trypsin digestion is generally carried out under conditions that maximize the digestion of the protein mixture with the use of a denaturant such as, but not limited to, 6 M guanidine chloride or 8 M urea, and by reduction and alkylation. In one example, to accomplish a complete digestion, trypsin (at a trypsin: sample protein ratio of about 1:100 is added to a sample, and the digestion is carried out for a total of about 24 hours. In some cases, trypsin is added to the sample in two aliquots at about a 1:50 ratio of protein substrate:trypsin (mass ratio). The trypsin is added to a sample that is to be analyzed further, e.g., a fraction eluted from an M-LAC procedure (flow-through fraction or bound fraction). The second aliquot of trypsin (at a ratio of about 1:50) can be added to the sample any time after the first, e.g., at 12 hours, 16 hours, 18 hours, or 24 hours.

Peptides from tryptic digests can be separated using methods known in the art. For example, capillary or nanoflow liquid chromatography electrospray ionization mass spectrometry (LCMS) can be used with a 75 μm reversed-phase capillary column or with other diameters, such as 150 μm, 300 μm, or 500 μm ID and/or a high performance liquid chromatography (HPLC) packing material that give good recoveries and separation of low level tryptic samples. In general, the peptides are identified by MS/MS fragmentation in an ion trap. It has been found that the linear ion trap (LTQ; Thermo Electron Corp., Austin, Tex.) results in about a two-fold increase in the number of peptide identifications. An alternative approach uses spotting or streaking or collection of the effluent in a 96 well plate or some other fraction collection device. The sample spot or streak is then dried down or physically admixed with a suitable matrix for matrix assisted laser desorption ionization (MALDI) and then measured typically by ion trap or time of flight mass spectrometry.

Peptide sequences are identified using algorithms available in the art, (e.g., the SEQUEST algorithm that is incorporated in BioWorks software, Version 3.1, ThermoFinnigan). In a non-limiting experiment, only peptides resulting from tryptic cleavages are searched. The protein identification is made based on the corresponding peptide identification. The SEQUEST search results are then exported to an Excel file and a comparison is made among the samples without M-LAC process, M-LAC elution, and M-LAC flow-through.

Glycoproteins and unbound fraction proteins can be isolated using M-LAC can be quantitated. The total amount of glycoprotein or glycoprotein and non-glycoprotein recovery can be assessed (e.g., as an amount relative to the total protein in sample prior to M-LAC) using methods known in the art such as Bradford protein assay, Lowry assay, biuret assay or bicinchonicnic acid (BCA) protein assay. In some methods, specific proteins are quantitated using methods known in the art. Such methods include, without limitation, immunochemical methods (e.g., ELISA or Western blot analysis), electrophoresis (e.g., one-dimensional gel electrophoresis or two-dimensional gel electrophoresis), and mass spectroscopy (e.g., of tryptic peptides).

Relative quantitation methods based on non-glycosylated tryptic peptides for mixtures of glycoproteins present in displacer fractions obtained from the multi-lectin columns. Table 1 shows typical results for the measurement of shifts of a glycoprotein in different displacer fractions after glycosidase digestions. Quantitation will also be performed by selected ion monitoring of a given glycoform in the new LT-FTMS system.

TABLE 1

Peak Areas of Selected Peptides of Glycoproteins
Captured by the M-LAC Procedure

| | |
|---|---|
| | Neuraminidized |
| Untreated sample | sample |

| | | | | | | | | |
|---|---|---|---|---|---|---|---|---|
| | | M/L | | Con | | | Con | |
| Protein | Selected Peptide | Signal | JAC | A | WGA | JAC | A | WGA |

| | | | | | | | | |
|---|---|---|---|---|---|---|---|---|
| TRFE | KPVEEYANCHLAR | 530.2 | 20 | 23 | 8.1 | 28 | 36 | 1.6 |
| | (SEQ ID NO:3) | | | | | | | |
| HPT | YVMLPVADQDQCIR | 854.6 | 10.1 | 39.3 | 236 | 12.6 | 46.9 | 163 |
| | (SEQ ID NO:4) | | | | | | | |
| A2HS | AQLVPLPPSTYVEFTVSGTDCVAK | 1290.32 | 4 | 2.6 | 1.7 | 7.4 | 2.6 | 0.9 |
| | (SEQ ID NO:5) | | | | | | | |
| APOH | ATVVYQGER | 512.3 | 2.6 | 3.5 | 1.8 | 3.8 | 8.4 | 0 |
| | (SEQ ID NO:6) | | | | | | | |
| AGP | EQLGEFYEALDCLR | 872.3 | 9.7 | 9.2 | 129 | 12.3 | 10.7 | 0 |
| | (SEQ ID NO:7 | | | | | | | |

After the use of neuraminidase to remove sialic acid residues from the glycosylation structures, the peak areas of the corresponding peptides were reduced in UGA fraction, and their peak areas were increased in either the Jacalin or Con A fraction, or both.

Glycoproteins isolated using M-LAC can be further fractionated, for example, by treating the glycoprotein fraction with a glycosidase that will induce specific glycosylation changes in glycoproteins susceptible to those glycosidases. The treated samples are then subjected to a second M-LAC column that can contain either the same lectins as are in the first column, or a second set of lectins. Glycosidase treatment exposes previous unexposed oligosaccharides and therefore permits a secondary level of fractionation of glycoproteins. The treated glycosylated proteins can either be eluted using a buffer that contains a displacement buffer appropriate for displacement of the glycoproteins from all of the M-LAC lectins, or the glycoproteins can be displaced using, e.g., only one displacement molecule in a wash. Sequential washes can also be used to separately elute those glycoproteins bound to each different lectin. Examples of glycosidases that can be used include neuraminidase, fucosidase, mannosidases, galactosidases, glucosidases, and others known in the art.

In some embodiments of the invention, oligosaccharides are removed from at least some members of a glycoprotein sample and analyzed. Such analyses are useful, e.g., for determining changes in the amount or type of oligosaccharide in a sample.

Glycoproteins in a glycoprotein sample can be deglycosylated using methods known in the art, e.g., hydrazinolysis or enzymatic removal (Fukuda, 1976, J. Biochem. (Tokyo) 80(6):1223-1232; El-Battari, 2003, Glycobiology 13(12):941-953). Commercially available methods for analyzing oligosaccharides can also be used, e.g., kits available from Marker Gene Technologies, Inc. (Eugene, Oreg.) and Glyko, Inc. (San Leandro, Calif.).

In certain methods of the invention, glycoproteins isolated using an M-LAC system are identified. Methods of identification are generally known in the art and include immunologic methods, gel electrophoresis methods such as two-dimensional electrophoresis, and mass spectroscopy methods including LC-MS/MA and LTQ FT-MSn, capillary electrophoresis with laser-induced fluorescence (CE-LIF), LC-MALDI-MS, or CE-MALDI-MS. Other useful methods include fluorescent probes, high pH anion-exchange chromatography with pulsed amperometric detection, normal phase high-performance liquid chromatography with fluorescent detection, surface plasmon resonance detection (Kuster et al., 2001, Proteomics 1(2):350-361; Kishino et al., 1997, J. Chromatogr. B. Biomed. Sci. Appl. 699(1-2):371-381; Mitchell et al., 2005 Proteomics Apr 19; [Epub]; Tran et al., 2001, J. Chromatogr. A 929(1-2):151-163; Duvinger et al, 2003, Biochemie 85:907).

In certain embodiments of the invention, the glycosylation structures of an obtained protein or protein fraction are assayed. In some cases, the assays are performed on a series of samples that are from, e.g., normal subjects (subjects that do not have a target disease) and subjects having a target disease, or from tissue culture cells subjected to various treatments or at various times during a treatment (e.g., with a cytotoxin). This is accomplished using methods known in the art, e.g., using antibodies that specifically bind to (are specific for) a selected protein sequence. A typical procedure uses magnetic beads (e.g., from Dynal (Oslo, Norway), SAM, 2.8 um) with the antibody covalently attached to the beads. The targeted glycoprotein is extracted from an M-LAC prepared protein fraction by incubating protein fraction with the antibody/bead conjugate and isolating the bound protein/antibody/bead complexes with a magnet (e.g., ThermoElectron Kingfisher). After washing to remove non-specifically bound material (e.g., using phosphate buffered saline containing 0.05% Tween-20 (PBSTween)), the captured glycoprotein is eluted by washing with 0.5 M acetic acid (pH 2.5). The eluted protein is concentrated, neutralized, and digested with trypsin. The resulting peptide peptides are analyzed (e.g., using LC/MS analysis in the linear ion trap).

The glycosylation structure can be determined using multiple rounds of MS/MS (MSⁿ) in a linear ion trap mass spectrometer, and quantitation of the individual glycoforms is achieved by analyzing MS peak intensity measurements of the different glycoforms in the FTMS. In another example, a glycopeptide fraction or non-fractionated sample is trypsinized and the resulting peptides are subjected to M-LAC. Thus, the glycosylated peptides resulting from the trypsin digestion can be isolated and these glycosylated peptides can be analyzed. The advantage of this method is that characterization of the structure of the glycopeptides by LC/MS is not complicated by the numerous non-glycosylated peptides that are generated when a sample of glycosylated proteins is trypsinized (Apffel et al., 1996, J. Chromatogr. A. 750(1-2):35-42; Apffel et al., 1996, J. Chromatogr. A. 732(1):27-42; Garcia et al., 1995, Anal. Biochem. 231(2):342-348, Otvos et al., 1992, J. Chromatogr. 599(1-2):43-49; and Xiong et al., 2002, J. Chromatogr. B. Analyt. Technol. Biomed. Life Sci. 782(1-2):405-418). Another advantage of this approach is that the reversed phase liquid chromatography (RPLC) gives partial separation between different glycostructures, which is helpful in terms of the subsequent MS analysis (Guzzetta et al., 1993, Anal. Chem. 65(21):2953-2962). The tryptic mapping approach generally leaves the glycan attached to the tryptic peptide and one can attempt to characterize the structure of both the peptide and glycan at the same time, typically by multiple rounds of MS fragmentation in a linear ion trap MS (Hirayama et al., 1998, Anal. Chem. 70(13):2718-25; Carr et al., 1993, Protein Sci. 2(2):183-196) or by the use of glycosidases (Zaia et al., 2001, Anal. Chem. 73(24):6030-6039; Iwase et al., 1999, J. Chromatogr. B. Biomed. Sci. Appl. 724(1): 1-7). This is facilitated by reversed phase LC (RPLC) in which the tryptic peptides can be concentrated, desalted and separated prior to analysis by mass spectrometry. Non-limiting examples of this approach include the characterization of plasminogen activator (PA) (two N-linked sites, one high in mannose and one of a complex type, Apffel et al., supra), tissue plasminogen activator (tPA, 3 N-linked glycosylation sites with complex sialylated structures, Chloupek et al., 1992, J. Chromatogr. 594:65-73) and erythropoietin (1995, Anal. Chem. 67(8):1442-1452).

The characterization and quantitation of glycosylation changes (with or without changes in the amount of protein) in different elution fractions provides a pattern or profile (“fingerprint”) of the glycoproteome in a biological sample. Methods known in the art can be used to characterize and quantitate the proteins in a sample. For example, ‘shotgun sequencing’ or an MuDPIT approach to identification of proteins using the tryptic digest of a sample is analyzed by LC/MS, and the identity of the peptide is determined by either MS fragmentation (MS/MS) or by accurate mass measurement (Washburn et al., 2001, Nat. Biotechnol. 19(3):242-247). In some cases, the captured proteins are identified by MS/MS of the non-glycosylated peptides, as such peptides are present in much higher levels and can be identified by database matching with an expected fragmentation pattern or accurate mass. To overcome the limitations of dynamic exclusion-based software, and the time constraints of a flowing system (a lower level peptide may not be detected in a single analysis because it is not selected for MS/MS fragmentation), triplicate LC/MS/MS measurements of the tryptic digest can be performed. The software systems used for transforming mass spectrometry information (e.g., MS/MS and accurate mass information) to a peptide identification are commercially available (e.g., SEQUEST (ThermoFinnigan), Mascot (Mass Spec UK), and SpectrumMill (Agilent, Palo Alto, Calif.)).

The methods can also be used to compare data from different LC/MS analyses. The certainty of an identification is increased by processing data using more than one software package since each has differences in the identification algorithm. For example, an initial identification is performed using the software BioWorks 3.1 (SEQUEST) to match MS/MS spectra to a Swiss-Prot human database. The search results are initially assessed using parameters that measure the quality of the spectra, e.g., the X_{corr} (cross correlation) and ΔCₙ (delta normalized correlation) scores. As a general rule, an X_{corr} value of greater than 2.5 for triply, 2.0 for doubly, and 1.5 for singly charged ions; ΔCₙ greater than 0.1; and Sp greater than 500, denotes a positive identification with greater than 90% confidence (Washburn et al., 2001, supra; Wu et al., 2003, J. Proteome Res. 2(4):383-93). The three matching factors (Sp, X_{corr}, and ΔCₙ) are used to construct a unified ranking score (Wu et al., 2003, supra) and a unified score value greater than 2400 again gives a confidence in the identification of greater than 90%. These filters can be combined (X_{corr} and the unified scores) to determine a confidence level of 99% or uncertainty of 0.01, and compare the peptide assignment from BioWorks with assignments from Mascot and SpectrumMill. Mascot matches observed m/z ions to ions generated from a random database and generates a probability score (Pappin et al., 1993, Curr. Biol. 3:327-332). PeptideProphet and ProteinProphet can also be used to interpret MS/MS data, as these programs differ from the others by placing additional match scores on the series of m/z ions obtained from a peptide fragmentation pattern (Yan et al., 2004, Mol. Cell. Proteomics 3(10):1039-1041.

Hybrid LTQ-FT-ICR (hybrid ion trap Fourier-transform ion cyclotron resonance mass spectroscopy) can be used to determine accurate mass measurements (within 2 ppm) on survey MS scans or MS/MS scans to provide further confidence to peptide identifications. Thus, the 2 ppm mass accuracy can be used as another filter to verify an assignment of identity. The exact mass measurement (e.g., monoisotopic mass) greatly narrows the possible candidates in a database search (e.g., from approximately 100,000 candidates with the ion trap to about 100 possible candidates with the FT-ICR). In some cases, to further confirm selected protein assignments, the molecular weight of intact proteins in the FTMS is determined. The mass of the multiply charged ions can be deconvoluted to provide the molecular weight of a protein by using a mathematical equation (Fenn et al., 1989, Science 246(4926):64-71). The accurate monoisotopic mass of a protein can be determined by a decluster software (e.g., Thrash, ThermoElectron, San Jose, Calif.), which predicts the monoisotopic mass from a resolved isotope pattern. In some cases, it may be desirable to remove the carbohydrate with glycosidase digestion before a mass measurement is made. Alternatively, the FT-ICR detector can be used in high resolution mode to determine the charge state of a protein from the resolved isotope pattern, not by deconvolution. This approach is particularly useful when these species are present in a complex mixture in which it may be difficult to determine discrete charge states at low mass resolution. FTMS can be used to measure the readily available monoisotopic masses of a protein fragment (e.g., obtained fragment ions from the collision-induced fragmentation in the linear ion trap) to search a database to identify the protein associated with the fragment. In general, these smaller ion fragments (e.g., less than 8 kD) in which the monoisotopic masses can be obtained without using any decluster software, are used first for matching the database. Once a protein is assigned using this match, the more complicated isotopic pattern ions (e.g., larger than 8 kD fragments) can be predicted from the assigned sequence. In some embodiments of the invention, a set of characteristic m/z ions are chosen to represent unique peptides from selected glycoproteins and monitor this set of m/z ion intensities in the FTMS to measure up-regulation or down-regulation of this specific set of proteins. The data from this approach can be correlated with data from two-dimensional gel studies of the samples.

Changes in glycosylation have been associated with disease. Therefore, M-LAC can be used for diagnosis, prognosis, and theranosis of such diseases. For example, rheumatoid arthritis (RA) and other rheumatic diseases are associated with a significant defect in the galactosyltransferase enzyme, which results in a profound change in the galactosylation of immunoglobulin G. (Axford, 1999, Biochim. Biophys. Acta 1455:219-29). Cancer malignancy, transformation and tumor progression have also been associated with by changes in cellular glycosylation (Schulenberg et al., 2003, J. Chromatogr. B. Analyt. Technol. Biomed. Life Sci. 793:127-139). M-LAC can also be used to assay advanced glycation end products (AGEs), which are the reactive derivatives of non-enzymatic glucose-macromolecule condensation product. Modified AGE proteins can accumulate in an age-dependent manner and contribute to age-related functional changes in vital organs.

The diagnostic methods described herein can also be used to identify subjects having, or at risk of developing, a disease or disorder associated with aberrant or unwanted glycosylation of a protein. As used herein, the term “unwanted” includes an unwanted phenomenon involved in a biological response such as pain or deregulated cell proliferation.

In one embodiment, a method of detecting a disease or disorder associated with aberrant or unwanted glycosylation is provided. In this method, a test sample is obtained from a subject and glycosylation of the sample (e.g., standards in the sample whose association with disease is predisposition to disease is known) is evaluated, wherein the presence of or level of at least one glycosylated protein is diagnostic for a subject having or at risk of developing a disease or disorder associated with aberrant or unwanted glycosylation. As used herein, a “test sample” refers to a biological sample obtained from a subject of interest. The sample can be, without limitation, a tissue, cell, or a biological fluid or secretion such as blood, serum, plasma, urine, semen, vaginal secretion, mammary secretion, cerebrospinal fluid, or saliva.

The compositions and methods described herein are useful for development of kits that can be used for characterization of the glycoproteome in a sample. Such kits include one or more M-LAC compositions (e.g., a column that contains at least two different lectins attached to a solid support such as agarose), at least two displacers, standards, and at least one buffer (e.g., wash buffer or displacement buffer). The kit can also include buffers, substrates, enzymes, chemicals and other compositions useful for further analysis of the glycoprotein fraction or unbound fraction.

Kits can also include components for sample preparation including, e.g., lysis buffer, dissociation reagent (e.g., for tissue preparations), and buffers that are optimized for isolation of specific sample types using a specified M-LAC system.

The methods, compositions, and kits described herein are useful for providing a platform for identifying global changes in major glycoproteins (e.g., at least 3, at least 5, at least 10, at least 25, or at least 50 glycoproteins) present in a sample, e.g., a serum sample or a cell lysate sample.

Kits can also contain one or more components for derivatization of a glycoprotein sample for CE-LIF analysis. A kit can also contain components for isolating glycoproteins using an M-LAC system for glycopeptide analysis using additional methods such as HPLC (e.g., preparative columns for glycopeptide analysis).

Samples can be analyzed in a multiplexed format for high throughput analysis. At least 6, at least 12, or at least 20 M-LAC columns, plates, or other format can be run using a rack/vacuum manifold. All columns are loaded, wash and eluted in parallel. The non-glycoproteins and glycoproteins are collected for analysis. The two fractions can be further loaded onto a C4, C8, or C18 solid phase extraction column and eluted with different concentrations of organic solvent, for example, acetonitrile. Depending on the depth of analysis required each column can be eluted with 3 or 5 or 10 different organic concentration wash steps into a 96 well plate. The sample can then analyzed, e.g., without further separation with direct infusion (for example, using a nanoelectrospray system such as a NanoMate™ (one nozzle per sample)). Any of the components used for such procedures can be included in an M-LAC kit. In some embodiments of the invention, components of an M-LAC kit that have been selected for use with particular commercially available system are supplied as supplemental kits for use with the commercially available system.

Multiple analysis kits are also provided by the invention. For example, the kit can contain one or more components for isolating glycoproteins using an M-LAC system and one or more components for processing the isolated glycoproteins for one or more methods, e.g., analysis for LC with ESI-infusion (e.g. Nanomate™ Autosampler interface) or CE with MALDI analysis), solid phase extraction (SPE) columns for ESI-infusion or MALDI analysis, and microtiter plate technology for use with, e.g., with a Beckman robotic workstation.

A single use M-LAC apparatus can be used to characterize the silent glycoproteome of a sample and to detect low level biomarkers (e.g., glycosylated biomarkers, non-glycosylated biomarkers, or both). Kits containing a single M-LAC apparatus (e.g., column) and at least two (e.g., three) lectins that are selected (e.g., optimized) for a particular application are therefore useful. Such kits can, optionally, include one or more of loading buffer, wash buffer, and lectin-specific displacers (e.g., supplied in elution buffer).

The invention also encompasses a kit that is useful for fractionating a non-glycoprotein fraction. Such a kit includes a depletion column, e.g., to remove albumin or other undesirable proteins and a multi-ligand column. For example a multi-ligand column containing a calmodulin bound to an agarose support can be used to specifically target Ca²⁺-calmodulin binding proteins (e.g., certain kinases, phosphatases, second messenger signalling proteins, cytoskeletal proteins, or muscle proteins) and a benzamidine bound to an agarose support to target specifically serine proteases. To use such a column, a sample is loaded onto the column and washed with an appropriate buffer (e.g., with 25 mM Tris, 150 mM NaCl, pH 7.4, 1 mM CaCl₂, and 1 mM MnCl₂). In some applications, HEPES is used instead of Tris. Two different displacers can be included in the kit, e.g., buffer containing 5 mM EGTA and 20 mM p-aminobenzamide. Other combinations of ligands can also be used in such applications, e.g., AMP, heparin, IMAC, calmodulin, and ATP. The combinations are chosen based on the type of biological information that is desired, e.g., to isolate a glycosylated and sulfated receptor a combination of a suitable lectin and histone can be used.

A representative kit of the invention includes at least one of a single M-LAC apparatus (e.g., column) and at least two (e.g., three) lectins selected for a particular application, loading buffer, wash buffer, and lectin displacers in appropriate buffers.

### EXAMPLES

The invention is further illustrated by the following examples. The examples are provided for illustrative purposes only. They are not to be construed as limiting the scope or content of the invention in any way.

### Example 1

### Multi-Lectin Affinity Chromatography Using Agarose-Bound Lectins

Materials

Human serum, dithiothreitol (DTT), iodoacetamide (IAA), sodium chloride, manganese chloride tetrahydrate, magnesium chloride, guanidinium hydrochloride, sodium azide, N-acetyl-glucosamine, galactose, methyl-α-mannopyranoside and calcium chloride were purchased from Sigma-Aldrich (St. Louis, Mo.). Ultra pure Tris and ammonium bicarbonate were purchased from ICN Biomedicals, Inc. (Aurora, Ohio). Agarose-bound Concanavalin A (Con A) with protein concentration of 6 mg lectin/ml gel and a binding capacity of more than 4 mg ovalbumin/mL gel, agarose-bound wheat germ agglutinin (WGA) with protein a concentration of 7 mg lectin/ml gel and binding capacity of 8 mg NGA/ml gel, agarose-bound Jacalin with a protein concentration of 4 mg lectin/ml gel and a binding capacity of more than 4 mg monomeric IgA/ml gel, agarose-bound peanut agglutinin (PNA) with a protein concentration of 5 mg lectin/ml gel and a binding capacity of more than 4 mg asialo-fetuin/ml gel, and agarose-bound lens culinaris agglutinin (LCA) with a protein concentration of 3 mg lectin/ml gel and a binding capacity of more than 3 mg mannosyl glycoprotein were obtained from Vector Laboratories (Burlingame, Calif.). Trypsin (sequence grade) was purchased from Promega (Madison, Wis.). Buffer A and Buffer B of the multiple affinity removal system were obtained from Agilent Technologies (Palo Alto, Calif.). NuPAGE® MOPS SDS running buffer and molecular mass standards were purchased from Invitrogen (Carlsbad, Calif.).

Preparation of Lectin Affinity Columns

Single-lectin, Con A, WGA, PNA, Jacalin, and LCA, affinity columns were prepared by adding 1 ml of corresponding agarose-bound lectin to empty PD-10 disposable columns (Amersham Biosciences, Piscataway, N.J.). The multi-lectin column was prepared by mixing 0.5 ml of agarose-bound Con A, 0.5 ml agarose-bound WGA, and 0.5 ml agarose-bound Jacalin in an empty PD-10 disposable column. The agarose gel was then fixed between two frits. The columns were either immediately used or stored in buffer (20 mM Tris, pH 7.4, 0.15M NaCl, 0.08% sodium NaN₃) at 4° C. The flow-through of the columns was gravity driven. The columns were not regenerated to avoid carry-over between different sample analyses.

Isolating Glycoproteins Using a Single-Lectin Affinity Column

To examine the spectrum of glycoproteins that can be isolated using single-lectin affinity columns, 100 μL human serum was diluted ten times with the equilibration buffer for that lectin (See Table 2) and loaded on the affinity column. After a 15 minute incubation, the unretained proteins were eluted with 8 ml of equilibration buffer, and the flow-through was collected. The captured glycoproteins were then released with 8 ml of the elution buffer specific for that lectin (Table 1), and the eluted fraction was collected. The flow-through and eluted fractions were concentrated using a 10 kD Amicon filter (4 ml, Millipore, Billerica, Mass.) and stored at −70° C. until further use.

TABLE 2
Binding buffers and elution solutions used for
single-lectin affinity columns

| | | Sugars Contained in |
|---|---|---|
| Lectin^{a} | Binding Buffer^{b} (pH 7.4) | Elution Buffer^{c} (pH 7.4) |
| Con A | 20 mM Tris, 0.15 NaCl, 1 mM | 0.5 M methyl-α- |
| | Ca²⁺, 1 mM Mn²⁺ | D-mannopyranoside |
| WGA | 20 mM Tris, 0.15 NaCl | 0.5 M N-acetyl-glucosamine |
| JAC | 20 mM Tris, 0.15 NaCl | 0.8 M galactose |
| PNA | 20 mM Tris, 0.15 NaCl, 1 mM | 0.5 M lactose |
| | Ca²⁺, 1 mM Mg²⁺ | |
| LCA | 20 mM Tris, 0.15 NaCl, 1 mM | 0.5 M methyl-α- |
| | Ca²⁺, 1 mM Mn²⁺ | D-mannopyranoside |

^{a}The lectin immobilized in a single-lectin column.
^{b}Binding buffer was used to prepare samples and remove non-specifically bound proteins
^{c}Elution buffer contained 20 mM Tris and 0.5 M NaCl.

Isolation of Glycoproteins Using a Multi-Lectin Affinity Column

To test the ability of a multi-lectin composition to effectively isolate glycoproteins, depleted serum samples (as described infra) or 200 μL of undepleted equilibration buffer (20 mM Tris, 0.15 M NaCl, 1 mM MnCl₂, and 1 mM CaCl₂, pH 7.4) to a volume of 2 ml, and were loaded on a multi-lectin affinity column. After a 15 minute reaction, the unbound proteins were eluted with 10 ml of equilibration buffer, and the captured proteins were released with 12 ml of elution (20 mM Tris, 0.5 M NaCl, 0.17 M methyl-α-D-mannopyranoside, 0.17 M N-acetyl-glucosamine and 0.27 M galactose, pH 7.4). The flow-through and eluted fractions were both collected and concentrated with 10 kD Amicon filters (15 ml, Millipore, Billerica, Mass.). The total amount of protein loaded on the column, the amount of protein collected in the flow-through and the amount of protein collected in eluted fraction were measured using a Bradford assay. The recovery from the multi-lectin column was calculated using the equation:

Recovery %=((flow-through+eluted protein)/total amount of protein loaded)×100.

The collected samples were stored at −70° C. until further testing. The same procedure was repeated using undepleted human serum including packing a multi-lectin affinity column.

To fractionate the proteins captured by the multi-lectin column, proteins bound to Jacalin lectin were first released with 4 ml of 0.8 M galactose in 20 mM Tris buffer pH 7.4, containing 0.15 M NaCl. Then, Con A selected proteins were released with 4 ml of 0.5 M methyl-α-D-mannopyranoside in a 20 mM Tris buffer, pH 7.4 containing 0.15 M NaCl. Finally, the WGA selected proteins were released with 4 ml of 0.5 M N-acetyl-glucosamine in 20 mM Tris buffer, pH 7.4 containing 0.15 M NaCl. The three fractions were concentrated with a 10 kD Amicon filter (4 ml capacity). The collected samples were stored at −70° C. until further testing.

Human Serum Depletion

A multiple immunoaffinity column (4.6×100 mm; Agilent Technologies, Palo Alto, Calif.) was used to remove albumin, IgG, antitrypsin, IgA, transferrin, and haptoglobin from human serum. The depletion procedure was performed on a HP 1090 LC system (Hewlett Packard, Palo Alto, Calif.). Briefly, human serum was diluted five times with Buffer A of the multiple affinity removal system and injected on the depletion column with an injection volume of 100 μL. Then the unbound serum proteins were eluted with Buffer A at a flow rate at 0.25 ml/min, and the flow through was collected. The column was then regenerated with Buffer B of the multiple immunoaffinity removal system before the next injection. The flow-through was concentrated with a 10 kD Amicon filter (15 ml capacity). A sample with approximately 1.3 mg of depleted serum proteins (compared with approximately 6.5 mg of undepleted human serum protein) was diluted to a volume of 2 ml with the multi-lectin binding buffer, and was further fractionated using the multi-lectin affinity column with the procedure described supra.

SDS-PAGE

The glycoprotein fractions isolated from the fractionation of serum on the single-lectin affinity columns and the corresponding human serum sample were analyzed on a NuPAGE® 4-12% bis-Tris gel (1.0 mm×10 well) (Invitrogen, Carlsbad, Calif.) with loading amount of 15 μg of protein for each fraction. The two glycoprotein fractions isolated from the single step elution of the multi-lectin affinity columns, the flow-through fraction from one multi-lectin column and set of three glycoprotein fractions from the use of different displacers on the multi-lectin affinity column were also analyzed on the NuPAGE® system (15 μg in each case). The proteins were resolved with the NuPAGE® MOPS SDS running buffer in a Novex® Mini-Cell system (Invitrogen) at 200 volts (PowerPac™ power supply, Bio-Rad, Hercules, Calif.). The proteins were visualized by staining with a glycoprotein detection kit (Sigma, St. Louis, Mo.) containing Schiff's reagent, which is a specific stain for glycoproteins. The staining was performed using the manufacturer's suggested protocol. To ensure the specificity of the staining, horseradish peroxidase, a glycoprotein, was analyzed on the same gel as positive control and a series of non-glycosylated molecular mass standards were used as negative control for the staining procedure.

Tryptic Digestion

The glycoprotein fractions (100 μg) from single- and multi-lectin affinity columns and the flow-through fraction from a multi-lectin column were digested with trypsin, using a standard procedure. Proteins were denatured with 6 M guanidine chloride in 0.1 M ammonium bicarbonate buffer, pH 8 and reduced by incubating with 5 mM DTT at 75° C. for one hour, then alkylated for two hours with 0.02 M iodoacetamide (IAA). The samples were solvent exchanged with a 10 kD Amicon filter (0.5 ml capacity, Millipore, Billerica, Mass.). The samples were then adjusted with 0.1 M ammonium bicarbonate buffer to a protein concentration of 0.5 mg/ml. Next, 1 μg trypsin was added to each sample and incubated at ambient temperature overnight. For complete digestion, another aliquot of 1 μg trypsin was added, and the digestion was continued for a total of 24 hours.

LC/MS/MS

Trypsin digested peptides were separated on a C18 capillary column (Biobasic C-18, Thermo, 180 μm×10 cm) using a ProteomeX™ system (ThermoFinnigan, San Jose, Calif.). The flow rate was maintained at 2 μL/min. The gradient was started at 5% acetonitrile (ACN) with 0.1% formic acid and a linear gradient to 40% ACN was achieved in 120 min, then ramped to 80% ACN in five minutes and kept at 80% ACN for 20 minutes to wash the column. Then, 15 μL of each sample containing 3.5 μg of protein was injected on the column from a Surveyor autosampler (ThermoFinnigan, San Jose, Calif.) using the no-waste injection mode. The resolved peptides were analyzed on an LCQ™ DECA XP ion trap mass spectrometer (i.e., an electrospray ionization/ion trap mass spectrometer; ThermoFinnigan, San Jose, Calif.) with an ESI (electrospray ionization) ion source. The temperature of the ion transfer tube was controlled at 185° C. and the spray voltage was 3.3 kV. The normalized collision energy was set at 35% for MS/MS. Data dependent ion selection was monitored to select the most abundant four ions from a MS scan for MS/MS analysis. Dynamic exclusion was continued for duration of five minutes.

Bioinformatics

Peptide sequences were identified using SEQUEST algorithm (Version C1) incorporated in BioWorks, Inc. software (Version 3.1) (ThermoFinnigan). Only peptides resulting from tryptic cleavages were searched. The SEQUEST results were filtered by Xcorr (which is used for determining correlations) vs. charge state. Xcorr was used for a match with 1.5 for singly charged ions, 2.0 for doubly charged ions, and 2.5 for triply charged ions. The protein identification was made based on the corresponding peptide identification. In this research, proteins with 2 or more peptide identifications were considered as positive identifications. Other search engines such as Mascot (a search engine that uses mass spectrometry data to identify proteins from primary sequence databases; Matrix Science Inc, Boston, Mass.), Spectrum Mill (Agilent Technologies), PeptideProphet™ (peptideprophet@sourceforge.net) can be used.

Proteins Captured by a Single-Lectin Affinity Column

To overcome the inability of a single lectin to completely capture a glycoprotein sample, five commonly used lectins, Con A, WGA, Jacalin, LCA and PNA, were examined separately for the capture of glycoproteins from human serum. Each lectin was immobilized on agarose and was separately packed in columns through which flow was driven by gravity. An equivalent sample of human serum was loaded on to each of these single-lectin affinity columns, and the captured proteins were eluted with a displacer (Table 1) that is specific for the corresponding lectin. After being concentrated, the captured protein samples were analyzed on SDS-PAGE and visualized using Schiff's reagent, which specifically stains glycoproteins (FIG. 1, lane 2 to lane 6). On the same gel, a positive control (FIG. 1, lane 1), horseradish peroxidase (glycoprotein), and a negative control (FIG. 1, lane 8) containing nonglycosylated protein molecular weight standards, were stained with the Schiff's reagent. In this case the glycoprotein standard developed color while the negative control did not stain, which confirmed the specificity of the Schiff's reagent for glycoproteins.

The groups of proteins captured by the different lectins had different SDS-PAGE profiles, although they were captured from the same human serum sample (FIG. 1). Some gel bands were detected in only one sample, such as band “a” in the lane of Con A bound proteins and band “c” in the lane of WGA bound proteins. Some bands had different staining intensities among the different samples although they migrated with the same approximate molecular weight. Examples are band “b” in the lane of Con A bound proteins and band “d” in the lane of Jacalin bound proteins. Based on the staining intensity, peanut agglutinin (PNA) did not capture as much glycoprotein from serum as the other four lectins. In addition, examination of gels corresponding to the Schiff's reagent-stained gels that were visualized with a Coomassie stain suggested that there was more nonspecific binding with both the PNA and LCA columns than with the other three lectins (Con A, WGA, and Jacalin) because more bands stained with Coomassie than with Schiff's reagent. An unfractionated serum sample was also analyzed on gels (e.g., FIG. 1, lane 7) and showed that the serum proteins were not as strongly stained by the Schiff's reagent relative to the lectin fractions. This is due to the presence of large amounts of non-glycosylated proteins such as albumin in the serum sample, therefore the glycosylated proteins represented only a small fraction of the total protein present in the unfractionated sample. These SDS-PAGE results indicated that each lectin can enrich specific sets of glycoproteins with some of overlap glycoprotein specificities. Also, as expected, none of the individual lectins could achieve a complete capture of all glycoproteins from a human serum sample.

Isolation of Glycoproteins Using a Multi-Lectin Affinity Column

A multi-lectin affinity column was prepared as described above from a physical mixture of immobilized Con A, WGA, and Jacalin lectins, and the column was used to enrich glycoproteins from human serum. These lectins were selected because their affinities cover most of the common sugar residues present in the O- and N-linked glycans that are contained in serum proteins. PNA and LCA were not included in the multi-lectin column because their specificities are similar to Jacalin and Con A, respectively, and their use resulted in more non-specific binding compared with the three selected lectins.

In these experiments human serum was loaded on the multi-lectin column. After washing out any unbound proteins (termed the ‘flow-through’ fraction), the captured glycoproteins (termed ‘bound’) were eluted with a specific displacer. The collected fractions were then digested with trypsin and analyzed with capillary reversed phase liquid chromatography (LC) with tandem mass spectra (LC-MS/MS) detection. The proteins were identified from the LC-MS/MS data using the SEQUEST algorithm [A]. In addition, the SEQUEST rank of each identified protein was used as an indication of the relative quantities of a given set of proteins. The specificity of this multi-lectin column was examined by an analysis of the glycosylation patterns of proteins identified in the bound (captured proteins) fraction (Table 3). Of the 51 proteins identified (with 2 or more peptide identifications) from the captured protein fraction, 50 were glycoproteins (including subunits of glycoproteins, such light chains of immunoglobulins), according to the Swissprot database (http://us.expasy.org/sprot/). Albumin, the exception, is not glycosylated but was found at low levels in the bound fraction (307 vs. 22 hits in the flow-through vs. bound fraction) indicating that albumin was largely not retained by the multi-lectin column. The presence of small amounts of albumin could either be due to a low level of non-specific binding or due to the formation of complexes with glycoproteins, such as IgA and IgG (Baumstark, 1983, Prep. Biochem. 13:15).

TABLE 3
Proteins Captured from Human Serum Using Multi-Lectin Affinity Column

| | | | Hits | Hits | |
|---|---|---|---|---|---|
| Rank^{a} | GI | Reference | (EL)^{b} | (FL)^{c} | Glycoprotein^{d} |
| 1 | A2MG | ALPHA-2-MACROGLOBULIN | 86 | 0 | Yes |
| 2 | TRFE | SEROTRANSFERRIN | 61 | 14 | Yes |
| 3 | HPT2 | HAPTOGLOBIN-2 | 60 | 0 | Yes |
| 4 | HPTR | HAPTOGLOBIN-RELATED PROTEIN | 60 | 0 | Yes |
| 5 | A1AT | ALPHA-1-ANTITRYPSIN | 53 | 7 | Yes |
| 6 | HEMO | HEMOPEXIN (BETA-1B-GLYCOPROTEIN) | 51 | 0 | Yes |
| 7 | ALC1 | IG ALPHA-1 CHAIN C REGION | 42 | 1 | Yes |
| 8 | CO3 | COMPLEMENT C3 | 34 | 0 | Yes |
| 9 | KAC | IG KAPPA CHAIN C REGION | 34 | 14 | Yes |
| 10 | APA1 | APOLIPOPROTEIN A-I | 24 | 17 | Yes |
| 11 | ALBU | SERUM ALBUMIN | 22 | 307 | No |
| 12 | AACT | ALPHA-1-ANTICHYMOTRYPSIN | 19 | 0 | Yes |
| 13 | A2HS | ALPHA-2-HS-GLYCOPROTEIN | 18 | 0 | Yes |
| 14 | MUC | IG MU CHAIN C REGION | 16 | 0 | Yes |
| 15 | ITH1 | INTER-ALPHA-TRYPSIN INHIBITOR HEAVY CHAIN | 14 | 0 | Yes |
| 16 | PZP | PREGNANCY ZONE PROTEIN | 14 | 0 | Yes |
| 17 | CERU | CERULOPLASMIN | 13 | 0 | Yes |
| 18 | CFAH | COMPLEMENT FACTOR H | 13 | 0 | Yes |
| 19 | CO4 | COMPLEMENT C4 | 12 | 0 | Yes |
| 20 | HRG | HISTIDINE-RICH GLYCOPROTEIN | 12 | 0 | Yes |
| 21 | GC1 | IG GAMMA-1 CHAIN C REGION | 12 | 19 | Yes |
| 22 | GC4 | IG GAMMA-4 CHAIN C REGION | 11 | 12 | Yes |
| 23 | A1AG | ALPHA-1-ACID GLYCOPROTEIN 1 | 11 | 0 | Yes |
| 24 | ITH2 | INTER-ALPHA-TRYPSIN INHIBITOR HEAVY CHAIN H2 | 11 | 0 | Yes |
| 25 | VTNC | VITRONECTIN | 9 | 0 | Yes |
| 26 | A1AH | ALPHA-1-ACID GLYCOPROTEIN 2 | 8 | 0 | Yes |
| 27 | APA2 | APOLIPOPROTEIN A-II | 7 | 6 | Yes |
| 28 | LAC | IG LAMBDA CHAIN C REGIONS | 7 | 11 | Yes |
| 29 | CLUS | CLUSTERIN | 7 | 0 | Yes |
| 30 | A1BG | ALPHA-1B-GLYCOPROTEIN | 6 | 0 | Yes |
| 31 | CFAB | COMPLEMENT FACTOR B | 6 | 0 | Yes |
| 32 | APOH | BETA-2-GLYCOPROTEIN-I | 6 | 0 | Yes |
| 33 | PLMN | PLASMINOGEN | 6 | 0 | Yes |
| 34 | APD | APOLIPOPROTEIN D | 6 | 0 | Yes |
| 35 | HBB | HEMOGLOBIN BETA CHAIN | 5 | 0 | Yes |
| 36 | KNG | KININOGEN | 5 | 0 | Yes |
| 37 | ANT3 | ANTITHROMBIN-III | 5 | 0 | Yes |
| 38 | ITH4 | INTER-ALPHA-TRYPSIN INHIBITOR HEAVY CHAIN H4 | 4 | 0 | Yes |
| 39 | IC1 | PLASMA PROTEASE C1 INHIBITOR | 4 | 0 | Yes |
| 40 | AMBP | AMBP PROTEIN | 3 | 0 | Yes |
| 41 | C4BP | C4B-BINDING PROTEIN ALPHA CHAIN | 3 | 0 | Yes |
| 42 | SAMP | SERUM AMYLOID P-COMPONENT | 3 | 0 | Yes |
| 43 | HBA | HEMOGLOBIN ALPHA CHAIN | 3 | 0 | Yes |
| 44 | C1QA | COMPLEMENT C1Q SUBCOMPONENT | 3 | 0 | Yes |
| 45 | KV3G | IG KAPPA CHAIN V-III REGION | 2 | 0 | Yes |
| 46 | HV3P | IG HEAVY CHAIN V-III REGION | 2 | 0 | Yes |
| 47 | HV1F | IG HEAVY CHAIN V-I REGION | 2 | 0 | Yes |
| 48 | THRB | PROTHROMBIN | 2 | 0 | Yes |
| 49 | LV3B | IG LAMBDA CHAIN V-III REGION | 2 | 0 | Yes |
| 50 | APC3 | APOLIPOPROTEIN C-III | 1 | 0 | Yes |
| 51 | APB | APOLIPOPROTEIN B-100 | 1 | 0 | Yes |

^{a}The rank is related to the probability of the MS assignment.
^{b}The number of peptides identified for a given protein captured by multi-lectin column.
^{c}The number of peptides identified in the protein in the flow-through fraction from multi-lectin column. 0 stands for not detected.
^{d}Whether the protein is or is not a glycoprotein or a subunit of a glycoprotein is derived from the Swissprot database.

The efficiency of this multi-lectin affinity column was further shown by the absence of a majority of the captured glycoproteins (41/51) in the flow-through fraction. In the case of three abundant glycoproteins (serotransferrin, alpha-1-antitrypsin, Ig alpha-1-chain c region) there was a significant enrichment in the bound fraction relative to the flow-through fraction based on the number of peptide identifications for each protein (Table 3). The incomplete binding of these proteins to the multi-lectin column could be due the presence of non-glycosylated isoforms in these proteins. Note that the capacity of the column is greater than the amount of protein loaded. In addition, the 1D-gel of the bound (FIG. 1, lane 6) and the flow-through (FIG. 1, lane 7) fractions showed that the bound fraction had a more intense and discrete banding pattern of glycoproteins relative to the flow-through fraction.

To demonstrate reproducibility of the multi-lectin affinity chromatography procedure for glycoprotein enrichment, the procedure was repeated (including packing of another multi-lectin column). The bound sample (glycoproteins captured by the second column) was analyzed by SDS-PAGE (FIG. 1, lane 5). The replicate showed a similar separation profile to that obtained with the first multi-lectin column (FIG. 1, lane 6). The LC/MS/MS analysis identified 50 proteins in the captured fraction and included 49 glycoproteins. Of these 50 proteins, 47 were in common with the glycoprotein fraction in the first experiment (46 out of these 47 proteins were glycoproteins, see Table 4) with albumin again as the only outlier.

TABLE 4
Number of Proteins Identified in Two Elution Samples Captured
from Replicate Multi-Lectin Affinity Columns^{a}

| Elution Sample^{b} | Protein ID (>2 hits)^{c} | Glycoproteins^{d} |
|---|---|---|
| 1 | 51 | 50 |
| 2 | 50 | 49 |
| Proteins in Common^{e} | 47 | 46 |

^{a}Proteins captured by the multi-lectin column were trypsin digested and analyzed by LC/MS/MS. The proteins were identified by using SEQUEST database search.
^{b}The glycoprotein isolation experiment was repeated including packing a replicate multi-lectin column. Therefore two elution samples were collected.
^{c}Number of proteins identified with two or more than two peptide identification.
^{d}Number of glycoproteins in the identified proteins with two or more peptide identification.
^{e}Number of proteins found in both elution samples

The rank of identified proteins as determined by the SEQUEST algorithm can represent an approximate relative abundance of the protein in the sample (see Table 5 for a comparison of relative abundances of the proteins). In general, the SEQUEST algorithm parameters are set conservatively to reduce the number of false positive identifications. This ranking is related to a preliminary score that is calculated based on the set parameters and this score is related to the number of ions in the MS/MS spectrum.

TABLE 5
Rank Differences for Same Proteins Identified in Two Elution Samples
Captured by Replicate Multi-Lectin Affinity Columns^{a}

| | Rank difference^{b} | | |
|---|---|---|---|
| | ≦5^{c} | ≦10^{d} | ≦0^{e} |
| Number of proteins^{f} | 38 | 46 | 50 |
| % of total proteins^{g} | 75 | 90 | 100 |

^{a}The rank of the protein can represent the approximate relative abundance of the protein in the sample.
^{b}The difference in rank of the same protein in the two elution samples.
^{c}The rank difference was equal or less than 5.
^{d}The rank difference was equal or less than 10.
^{e}The rank difference was equal or less than 20.
^{f}The number of proteins identified with two or more peptide identifications.
^{g}The percentage of the number of proteins in each rank relative to the total number of proteins identified with two or more peptide identifications.

Among the proteins identified in both elution samples, 38 out of 50 proteins (75%) were ranked in a consistent manner (difference of <5) and 46 proteins (90%) were found with a rank difference less or equal to 10. Finally all of the 50 proteins were identified with rank difference less than 20. Here, conservative protein identifications were used, namely 2 or more tryptic peptide hits with a conservative MS filter (described supra). The reproducibility of the ranking was found to be improved by the use of these conservative filters.

The amount of protein loaded on the multi-lectin column and the amount of protein recovered (flow-through and bound) were measured using a Bradford assay.

A study of the recovery achieved with the multi-lectin affinity column showed that about 0.6 mg of glycoproteins were enriched in the bound fraction from a total of approximately 6.7 mg of serum protein (i.e., about 11% of serum proteins bound to the column) and about 5.0 mg of protein was collected in the flow-through fraction. Therefore, the recovery of this multi-lectin enrichment procedure was about 84%. The sample loss was primarily due to the 10 kD MWCO filtration step used to concentrate the fractions (supra). From these data it was concluded that at least 10% of human serum proteins were glycosylated, considering potential sample losses during the process, while most of the large amount of non-glycosylated material was due to albumin.

### Example 2

### Isolating Glycoproteins from Depleted Serum

To improve dynamic range of glycoprotein identifications on a multi-lectin column, human serum was first depleted with a multiple immunoaffinity removal column (as described herein) that specifically removes albumin, IgG, IgA, antitrypsin, transferrin, and haptoglobin. This depletion step removed approximately 80% of serum proteins and yielded a sample with about 1.3 mg of depleted serum proteins (from 6.5 mg of serum protein). The depleted sample was then loaded onto the multi-lectin column and a 0.6 mg glycoprotein fraction was captured (50% of the total protein present in the depleted serum sample). In the LC/MS/MS results, 42 proteins were identified in this captured fraction and the six high abundance proteins that were expected to be removed (i.e., depleted from the glycoprotein fraction) were indeed, not observed. The 42 proteins observed in the depleted sample were also observed in the analysis of the original serum sample. Despite the lack of increase in the number of proteins in the lectin-selected serum sample compared to a lectin-selected serum sample after depletion of the six most abundant proteins, the glycoproteins were observed with greater sequence coverage in the latter experiment. The lack of increase in dynamic range may be due to the fact that the lectin column itself represents a significant depletion step. Another factor is that there are many more abundant proteins than the few depleted in this study, for example alpha-2-macroglobulin was present in large amounts in the purified glycoprotein sample.

### Example 3

### Protein Fractionation on a Multi-Lectin Column Using a Sequential Displacement

The proteins captured by the multi-lectin column from non-depleted human serum were fractionated by sequential use of displacers specific for each lectin. The sequence of specific displacers was as follows: Jacalin, Con A, and finally, WGA lectin. The three fractions containing the eluted glycoproteins were analyzed by SDS-PAGE (FIG. 1, lanes 2, 3, and 4). These data showed that each of the three fractions had a significantly different 1D-gel profile (FIG. 1, lanes 2, 3, and 4). The three fractions were also analyzed by LC/MS/MS and Table 6 lists the distribution of the proteins in the three displacement fractions with approximate abundance (as measured by LC-MS/MS of each protein (+ to ++++) observed in a given fraction.

TABLE 6
Significant Proteins Eluted by Specific Displacers for a
Specific Lectin in a Multi-Lectin Column

| | | Abundance^{a} | Abundance | Abundance |
|---|---|---|---|---|
| GI | Reference | (Jacalin)^{b} | (ConA)^{c} | (WGA)^{d} |
| A1AT | Alpha-1-antitrypsin | + | + | |
| A1BG | ALPHA-1B-GLYCOPROTEIN | | +++ | |
| A2HS | ALPHA-2-HS-GLYCOPROTEIN | ++ | | +++ |
| A2MG | ALPHA-2-MACROGLOBULIN | | + | + |
| AACT | ALPHA-1-ANTICHYMOTRYPSIN | | ++++ | |
| ALBU | SERUM ALBUMIN | + | + | ++++ |
| ALC1 | IG ALPHA-1 CHAIN C REGION | + | ++ | + |
| APA1 | APOLIPOPROTEIN A-I | | +++ | |
| APC3 | APOLIPOPROTEIN C-III | ++ | | |
| APE | APOLIPOPROTEIN E | +++ | | |
| CBP8 | CARBOXYPEPTIDASE N 83 KDA | | | ++ |
| | CHAIN | | | |
| CERU | CERULOPLASMIN | | ++++ | |
| CFAB | COMPLEMENT FACTOR B | | ++++ | |
| CFAH | COMPLEMENT FACTOR H | | ++ | |
| CO3 | COMPLEMENT C3 | | + | |
| GC1 | IG GAMMA-1 CHAIN C REGION | | +++ | |
| GC3 | IG GAMMA-3 CHAIN C REGION | ++++ | | |
| HEMO | HEMOPEXIN | | +++ | + |
| HPT2 | HAPTOGLOBIN-2 | +++ | ++ | + |
| HPTR | HAPTOGLOBIN-RELATED PROTEIN | ++ | ++ | + |
| IC1 | PLASMA PROTEASE C1 INHIBITOR | | | ++++ |
| ITH1 | INTER-ALPHA-TRYPSIN INHIBITOR | ++ | | +++ |
| | HEAVY CHAIN H1 | | | |
| ITH2 | INTER-ALPHA-TRYPSIN INHIBITOR | ++++ | | |
| | HEAVY CHAIN H2 | | | |
| MUC | IG MU CHAIN C REGION | | ++++ | ++ |
| PZP | PREGNANCY ZONE PROTEIN | | ++++ | ++ |
| TRFE | SEROTRANSFERRIN | ++++ | + | |
| VTNC | VITRONECTIN | | | ++ |

^{a}The relative abundance was defined by grouping the SEQUEST ranking (5 rankings per a group). Proteins ranked 1 to 5 have an abundance of +, ranked 6 to 10 have an abundance of ++, ranked 11 to 15 have an abundance +++, and ranked 16 to 20 have an abundance of ++++. The empty boxes denote protein rankings that were not in the top 20.
^{b}The abundance score of proteins identified in the Jacalin displacement fraction.
^{c}The abundance score of proteins identified in the Con A displacement fraction.
^{d}The abundance score of proteins identified in the WGA displacement fraction.

These results show that many glycoproteins were concentrated in a specific displacer fraction and this distribution could be correlated with known glycosylation structures (listed in the Swissprot database). Some proteins were eluted in all displacer fractions, indicating cross-reactivity with all three lectins that were selected for the separation. Examples of such proteins are IgA and haptoglobin. IgA has five O-linked glycosylation sites and two N-linked glycosylation sites. Because of the extensive O-glycosylation and its high abundance in human serum, IgA was one of the most abundant proteins in the Jacalin displacement fraction (ranked at position 1). Haptoglobin was highly enriched in the WGA displacement fraction, which may be related to a high abundance of N-acetylglucosamine (GlcNAc). Some proteins were found to be highly abundant in only one or two displacement fractions. Alpha-1-antitrypsin, which is normally N-glycosylated at three asparagine residues (residues 46, 83, and 247), was enriched in the Jacalin fraction as well as the Con A fraction, which may suggest the existence of O-linked glycosylation or galactosyl (β-1, 3) GalNAc residues in its carbohydrate moieties. In contrast, alpha-2-macroglobulin, which has eight N-linked glycosylation sites, was found to be highly abundant in both the Con A and WGA displacement fractions. The absence of this protein in the Jacalin fraction suggests the lack of O-linked glycosylation on this protein, particularly as it is a relatively high abundance protein in human plasma (2000 mg/L). Pregnancy zone protein was highly enriched in the WGA displacement fraction, although it is a relatively low abundance plasma protein (8 mg/L; Petersen et al., 1990, Clin. Lab. Invest. 50:479) and this result demonstrates the ability of the multi-lectin affinity column to enrich specific glycoproteins. Apolipoprotein E has only an O-linked glycosylation site (Thr 212) and was only found in the Jacalin fraction, which further indicated that the expected lectin specificity was indeed observed in the fractionation process. These results indicated that the distribution of a glycoprotein into each of the three fractions is determined to a large extent by the glycosylation pattern of the glycoprotein. In addition, albumin was found in all three displacer fractions, but with the abundance sequentially decreased, which suggests that the capture of albumin by the multi-lectin column was due to non-specific binding.

The reproducibility of the fractionation procedure is further established by observation of a similar distribution of the glycoproteins in the three displacement fractions with other plasma and serum samples.

### Example 4

### Glycoprotein Identification in Serum and Plasma Using M-LAC and RPLC-MS/MS

To demonstrate the usefulness of M-LAC as part of a method of identifying glycoproteins in a sample, human serum and plasma samples were isolated using M-LAC and tryptic digests of the isolated glycoproteins were used for protein identification.

Materials

Human plasma and serum samples were provided by the Human Proteome Organization (HUPO; McGill University and Génome Québec Innovation Centre Réseau Protéomique de Montréal; Montreal Proteomics Network, Montreal (QC), Canada). A total of twelve samples were used that were from the pools of three ethnic groups described as Caucasian American, African-American, and Asian-American. Three plasma samples and one serum sample from each ethnic group was tested. The plasma samples were collected in plasma tubes containing either sodium citrate, lithium heparin, or K₂EDTA as the anticoagulant using methods known in the art. Agarose bound lectins (Concanavalin A (Con A), wheat germ agglutinin (WGA), and Jacalin) were purchased from Vector Laboratories (Burlingame, Calif.).

Isolating Glycoproteins Using Multi-Lectin Affinity Columns

The preparation of multi-lectin affinity columns for M-LAC and the procedure of enriching glycoproteins from human serum and plasma were as described above. Briefly, multi-lectin columns were prepared by mixing equal amount of agarose-bound Con A, agarose-bound WGA, and agarose-bound Jacalin in an empty PD-10 disposable column. A 50 μL sample of serum or plasma was diluted with multi-lectin column equilibrium buffer (M-LAC binding buffer) (20 mM Tris, 0.15 M NaCl, 1 mM MnCl₂ and 1 mM, CaCl₂, pH 7.4) to a volume of 1 ml, and was then loaded on a newly packed multi-lectin affinity column. After a 15 minute incubation, the unbound proteins were eluted with 10 ml of M-LAC binding buffer, and the captured proteins were released with 12 ml of displacer solution (20 mM Tris, 0.5 M NaCl, 0.17 M methyl-α-D-mannopyranoside, 0.17 M N-acetyl-glucosamine, and 0.27 M galactose, pH 7.4). The multi-lectin affinity column captured fraction was collected and concentrated using 15 ml, 10 kD Amicon filters (Millipore, Billerica, Mass.).

Analysis of Glycoproteins Using LC-LCQ MS

One hundred micrograms of glycoproteins collected from M-LAC were digested with trypsin, using a procedure known in the art. Briefly, proteins were denatured with 6 M guanidine chloride in 0.1 M ammonium bicarbonate buffer, pH 8. Immediately after adding the protein to the buffer, the reduction reaction was initiated. Samples were reduced by incubating the samples with 5 mM DTT at 75° C. for one hour, then alkylated for two hours with 0.02 M iodoacetamide (IAM). The samples were solvent exchanged with a 10 kD Amicon filter (0.5 ml capacity, Millipore, Billerica, Mass.). The samples were then adjusted with 0.1 M ammonium bicarbonate buffer to a protein concentration of 0.5 mg/ml. One microgram of trypsin was added to each sample and the samples were incubated overnight at ambient temperature. For complete digestion, another aliquot of 1 μg trypsin was added, and the digestion was continued for a total of 24 hours.

The peptides were separated on a C18 capillary column (packed in-house, 150×0.075 mm) using a Surveyor LC pump (ThermoFinnigan, San Jose, Calif.). The flow rate was maintained at 300 nl/min. A gradient was started at 5% acetonitrile (ACN) with 0.1% formic acid and a linear gradient to 40% ACN was achieved in 165 minutes, then ramped to 60% ACN in 20 minutes and to 90% in the following 10 minutes. Ten microliters of each sample containing 2 μg of protein was injected onto the column from a Surveyor autosampler (ThermoFinnigan, San Jose, Calif.) using the full injection mode. The resolved peptides were analyzed on an LCQ DECA XP ion trap mass spectrometer (ThermoFinnigan, San Jose, Calif.) with an ESI ion source. The temperature of the ion transfer tube was controlled at 185° C. and the spray voltage was 2.0 kV. The normalized collision energy was set at 35% for MS/MS. Data-dependent ion selection was monitored to select the five most abundant ions from a MS scan for MS/MS analysis. Dynamic exclusion was continued for duration of two minutes.

Analysis of Glycoproteins Using LC-LTQ MS

The glycoproteins enriched from Caucasian-American serum samples were digested, and the digests were separated on a capillary column (Thermo Hypurity, C18, 150×0.075 mm) using an Ettan MDLC system from Amersham Biosciences (Piscataway, N.J.). The separation gradient was similar as that described in above, except that the starting point was 0% B (0.1% formic acid in 100% acetonitrile) due to the use of a trap column for desalting the sample before it was deliver to the mass spectrometer (Michrom Bioresources, Inc.; Auburn, Calif.) in front of the separation column. The resolved peptides were analyzed on a LTQ mass spectrometer (ThermoFinnigan, San Jose, Calif.) with an ESI ion source. The temperature of the ion transfer tube was controlled at 185° C. and the spray voltage was 2.0 kV. The normalized collision energy was set at 35% for MS/MS. Data-dependent ion selection was monitored to select the five most abundant ions from a MS scan for MS/MS analysis. Dynamic exclusion was continued for duration of two minutes.

Peptide sequences were obtained by searching protein databases The proteins were identified using SEQUEST algorithm (Version C1) incorporated in BioWorks software (Version 3.1) (ThermoFinnigan, La Jolla, Calif., fields.scripps.edu/sequest) and the Swissprot Human Protein Database (au.expasy.org/sprot/). The database search was limited to only the peptides that would be generated by tryptic cleavage. The SEQUEST results were filtered by Xcorr vs. charge state. Xcorr was used for a match with 1.5 for singly charged ions, 2.0 for doubly charged ions, and 2.5 for triply charged ions. The protein identification was made based on the corresponding peptide identification.

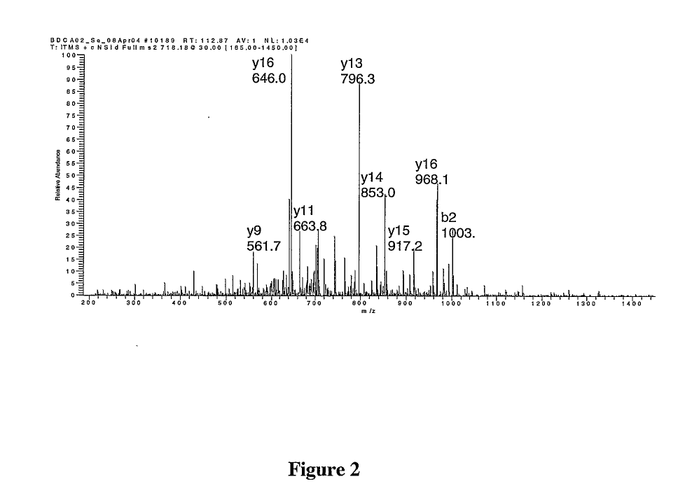

The tryptic digests of glycoproteins isolated from plasma or serum samples were analyzed on an ion trap mass spectrometer (LCQ-MS) as described above, and proteins with two or more peptide identifications from all 12 samples were considered to be positive identifications. Table 7, shown below, lists nine additional glycoproteins that were identified with only one peptide, but for which the identifications were subsequently confirmed by a separate analysis of the Caucasian-American serum sample using the more powerful linear ion trap (LTQ-MS). In this analysis 6 of these 9 proteins also had better quality identifications on LTQ (with 2 or more peptide IDs). The remaining three protein identifications were made with a single peptide ID on both the LTQ-MS and LCQ-MS studies, and were also confirmed by manual inspection of the peptide fragmentation spectra (See FIG. 2). In these spectra, the signals were observed with low noise levels and extensive b or y ion fragments. Therefore, these three proteins were evaluated to be positive identifications, and compilation of all these identifications gave a total of 158 glycoproteins. The identified glycoproteins were alpha-1-acid glycoprotein 1, alpha-1-acid glycoprotein 2, alpha1-antitrypsin, alpha-1-antitrypsin-related protein, alpha-1B-glycoprotein, clathrin coat assembly protein AP19, alpha-2-antiplasmin, leucine-rich alpha-2-glycoprotein, alpha-2-hs-glycoprotein, alpha-2-macroglobulin, alpha-1-antichymotrypsin, 5′-AMP-activated protein kinase, gamma-2, activator 1 140 kda subunit, acyl-COA dehydrogenase, (very-long-chain sp), afamin, putative fork head domain, ranscription F, Ig alpha-1 chain c region, Ig alpha-2 chain c region, AMBP protein, angiotensinogen, antithrombin-iii, apolipoprotein a-i, apolipoprotein a-ii, apolipoprotein b-100, apolipoprotein c-iii, apolipoprotein d, apolipoprotein e, beta-2-glycoprotein i, aspartyl/asparaginyl beta-hydroxylase, biliverdin reductase A, complement C1Q subcomponent, B chain, complement C1Q subcomponent, C chain, complement C1R component, C4B-binding protein alpha chain, CMP-N-acetylneuraminate-beta-galactosamide, adenylyl cyclase-associated protein 1, corticosteroid-binding globulin, ceruloplasmin, complement factor B, complement factor H, cystic fibrosis tansmembrane conductance, calcitonin gene-related peptide type 1 rec, serine/threonine-protein kinase CHK2, clusterin, rod cgmp-specific 3′,5′-cyclic phosphodies terase, complement C3, complement C4, complement C5, complement component C8 alpha chain, complement component C8 gamma chain, complement component C9, coatomer beta subunit, cytochrome p450 3A4, crumbs protein homolog 1, catenin delta-1, DNA fragmentation factor alpha subunit, DNA2-like homolog, double-stranded RNA-specific adenosine DEA, coagulation factor XI, complement factor H-related protein 1, complement factor H-related protein 3, fibrinogen alpha/alpha-E chain, fibrinogen beta chain, fibrogen gamma chain, fibronectin, Ig gamma-1 chain C region, Ig gamma-2 chain C region, Ig gamma-3 chain C region, Ig gamma-4 chain C region, g protein pathway suppressor 1, granzyme A, glutathione reductase, hemoglobin alpha chain, hemoglobin beta chain, hemoglobin gamma-A and gamma-G chains, hemopexin precursor (beta-1B-glycoprotein), heparin cofactor II, voltage-gated potassium channel, haptoglobin-2 precursor, haptoglobin-related protein, histidine-rich glycoprotein, heat shock 27 KDA protein, Ig heavy chain V-I region, Ig heavy chain V-III region, Ig heavy chain V-III region, Ig heavy chain V-III region, Ig heavy chain V-III region, Ig heavy chain V-III region, Ig heavy chain V-III region, Ig heavy chain V-III region, Ig heavy chain V-III region, plasma protease C1 inhibitor, immunoglobulin J chain, importin beta-2 subunit, inward rectifier potassium channel 4, inter-alpha-trypsin inhibitor heavy chain, inter-alpha-trypsin inhibitor heavy chain H, inter-alpha-trypsin inhibitor heavy chain, keratin, type i cytoskeletal 18, keratin, type i cuticular HA4, keratin, type i cuticular ha5, keratin, type i cuticular ha6, keratin, type i cuticular ha3-ii, Ig kappa chain c region, creatine kinase, kininogen, Ig kappa chain v-i region, Ig kappa chain v-i region, Ig kappa chain v-i region, Ig kappa chain v-i region, Ig kappa chain v-ii region, Ig kappa chain v-ii region, Ig kappa chain v-iii region, Ig kappa chain v-iii region, Ig kappa chain v-iii region, Ig kappa chain v-iii region hic, Ig lambda chain c regions, Ig lambda chain v-iii region, Ig lambda chain v-iv region, Ig lambda chain v-vi region, mitogen-activated protein kinase 7, DNA mismatch repair protein MSH3, Ig mu chain c region, myosin heavy chain, skeletal muscle, myosin heavy chain, nonmuscle type A, BCL2/adenovirus E1B 19-KDA protein-interac, NADH-ubiquinone oxidoreductase 39 KDA, phosphatidylinositol 3-kinase regulatory B, polycystin 2, plectin 1, plasminogen, serum paraoxonase/arylesterase 1, pregnancy zone protein, 60S ribosomal protein L32, 60S ribosomal protein L5, ryanodine receptor, (skeletal muscle), ryanodine receptor 2, serum amyloid P-component, senescence marker protein-30, SWI/SNF-related, matrix-associated, actin, symplekin, signal recognition particle 54 KDA protein, arginyl-TRNA synthetase, prothrombin, serotransferrin, lactotransferrin, trichohyalin, thyroid receptor interacting protein 7, troponin T, cardiac muscle isoforms, tetratricopeptide repeat protein 3, transthyretin, U2 small nuclear ribonucleoprotein auxillia, vitronectin, WEE1-like protein kinase, hypothetical protein kiaa0167, hypothetical protein kiaa0188, zinc finger protein 33A, zinc-alpha-2-glycoprotein,

TABLE 7
Protein Identifications Confirmed by LTQ Analysis^{a}

| ID^{b} | Protein^{b} | Rank^{c} | Hits^{d} |
|---|---|---|---|
| A2AP | ALPHA-2-ANTIPLASMIN | 40 | 7 |
| C1QB | COMPLEMENT C1Q SUBCOMPONENT, | 56 | 4 |
| | B CHAIN | | |
| CO8G | COMPLEMENT COMPONENT C8 GAMMA | 69 | 2 |
| | CHAIN | | |
| FA11 | COAGULATION FACTOR E XI | 58 | 4 |
| KV3F | IG KAPPA CHAIN V-III REGION | 74 | 2 |
| LV3B | IG LAMBDA CHAIN V-III REGION | 70 | 2 |
| CBG | CORTICOSTEROID-BINDING GLOBULIN | 84 | 1 |
| HV3A | IG HEAVY CHAIN V-III REGION | 92 | 1 |
| NUEM | NADH-UBIQUINONE OXIDOREDUCTASE | 182 | 1 |

^{a}The proteins identified with only one peptide ID in LC-LCQ MS were confirmed in the LC-LTQ MS analysis of Caucasian American serum sample.
^{b}Swissprot database code and protein name.
^{c}Rank form SEQUEST algorithm.
^{d}Number of ms/ms identifications in a given protein sequence.

In addition to providing improved protein identifications, the number of identified glycoproteins isolated from Caucasian-American serum sample was doubled by using LTQ and half of these proteins were identified with two or more peptides. These samples demonstrated a high degree of reproducibility for the HPLC separation. The improved proteomic analysis may be due both to the mass spectrometer used and improved chromatographic performance achieved with MDLC. The observed delay in time for the peptides to elute in the LC-LTQ experiment was due to a difference in starting conditions for the separation (0% instead of 5% B).

Comparison of Serum and Plasma Glycoproteomes

In each ethnic group sample, the proteins absent in serum, but present in more than one of the three plasma samples were identified. The reproducibility of the protein identifications among the three ethnic group samples was evaluated and used to construct a summary protein list and assess any observed differences between plasma and serum identifications.

A conservative set of criteria were used for protein identification, requiring identification based on at least two tryptic peptides combined with the use of a differential SEQUEST score of >20 in relative ranking for comparative studies of different glycoprotein samples. This approach has been validated by the exploration of false positives with independent criteria such the use of measured isoelectric point (pI) values and the measurement of peak areas of selected peptide ions. Using these evaluations, it was found that more protein identifications were made in the plasma samples compared to the serum samples. The major observed differences were the absence of proteins in the plasma sample that were present in the serum sample. For example, as expected, fibrinogen and plasminogen were present in plasma, but not present to any significant extent in serum (Table 8). These results demonstrate that serum preparations maintained the majority of the plasma glycoproteome, and so are suitable for many applications for analysis of the serum/plasma proteome. In addition, the identification of fibrinogen was improved in heparinized plasma in which fibrinogen had a higher rank and more peptide identifications. This indicates that, in some cases, heparin is better than EDTA and citrate for stabilizing plasma.

TABLE 8
Comparison of HUPO Human Serum and Plasma Samples

| | Sample Type | | | | | | | |
|---|---|---|---|---|---|---|---|---|
| | Rank^{a} | | | | Hits^{b} | | | |
| Ethnic Group | HEP | EDTA | CIT | Serum | HEP | EDTA | CIT | Serum |
| FIBRINOGEN ALPHA/ALPHA-E CHAIN | | | | | | | | [symbol] |
| Caucasian American | 11 | 14 | | | 42 | 18 | | |
| Asian American | 5 | | 54 | | 53 | | 1 | |
| African American | 8 | | | | 47 | | | |
| FIBRINOGEN BETA CHAIN | | | | | | | | [symbol] |
| Caucasian American | 10 | 15 | | | 42 | 17 | | |
| Asian American | 11 | | 36 | | 37 | | 3 | |
| African American | 9 | | | | 47 | | | |
| FIBRINOGEN GAMMA CHAIN | | | | | | | | [symbol] |
| Caucasian American | 9 | 12 | | | 45 | 23 | | |
| Asian American | 8 | 45 | 26 | | 49 | 3 | 5 | |
| African American | 7 | | | | 55 | | | |
| PLASMINOGEN | | | | | | | | [symbol] |
| Caucasian American | 58 | 50 | 32 | | 1 | 2 | 4 | |
| Asian American | 42 | 52 | 55 | | 4 | 2 | 1 | |
| African American | 40 | 52 | 32 | 56 | 4 | 2 | 3 | 1 |

^{a}Rank form SEQUEST algorithm.
^{b}Number of ms/ms identifications in a given protein sequence. [symbol]
: The concentration of the indicated protein was at a much lower level in serum than in plasma.

### Example 5

### Comparison of the Glycoproteomes Between Ethnic Samples

To determine whether the methods could be used to distinguish individuals and/or groups, glycoproteomes of the different ethnic samples were compared. The proteins that consistently had higher or lower levels (a difference in MS rank of more than 20) in all four types of plasma/serum samples relative to an individual ethnic group were considered to be either up- or down-regulated. This ranking requires a high degree of consistency between LC-MS/MS analyses and thus was applied only to samples run in a consecutive series. In addition the data sets included only high probability protein assignments.

The tested serum and plasma samples consisted of pools of one female and one male subject from three ethnic groups. Overall conclusions on the effect of ethnicity on the expression of the proteome cannot be made from such a limited sample set, however, an analysis provides an opportunity to explore the ability of the methods to analyze different samples. It was found that the Caucasian-American sample (a pooled sample from two individuals) had an increased level of angiotensinogen (AGT) and reduced level histidine-rich glycoprotein (HRG) relative to the other two samples, and vitronectin (VNT) was present at a lower level in the African-American sample (Table 9).

TABLE 9
Comparison among HUPO samples from three ethnic groups

| | Rank^{a} | | | Hits^{b} | | | Change of |
|---|---|---|---|---|---|---|---|
| Sample type | AA | AFA | CA | AA | AFA | CA | regulation |
| ANGIOTENSINOGEN (Up regulated in CA) | | | | | | | |
| Citrated Plasma | | | 45 | | | 2 | in CA |
| EDTA Plasma | 92 | | 42 | 1 | | 4 | [symbol] |
| Heparin Plasma | 37 | 46 | 41 | 5 | 3 | 4 | |
| Serum | 66 | 62 | 47 | 1 | 1 | 2 | |
| HISTIDINE-RICH GLYCOPROTEIN | | | | | | | |
| (Down Regulated in CA) | | | | | | | |
| Citrated Plasma | 50 | | | 1 | | | in CA |
| EDTA Plasma | 29 | 48 | 69 | 7 | 2 | 1 | [symbol] |
| Heparin Plasma | 36 | 30 | 40 | 5 | 8 | 4 | |
| Serum | 35 | 44 | | 4 | 2 | | |
| VITRONECTIN PRECURSOR (Up Regulated in AA) | | | | | | | |
| Citrated Plasma | 30 | 34 | 34 | 5 | 4 | 4 | in AFA |
| EDTA Plasma | 32 | | 60 | 7 | | 2 | [symbol] |
| Heparin Plasma | 33 | 39 | 48 | 6 | 2 | 3 | |
| Serum | 37 | | 63 | 4 | | 1 | |

^{a}Rank form SEQUEST algorithm.
^{b}Number of ms/ms identifications in a given protein sequence.

These data are consistent with a report that M235T, an AGT polymorphism, was associated with a stepwise increase in AGT levels in white subjects (Sethi et al., 2003, Arterioscler. Thromb. Vasc. Biol. 23:1269).

Histidine-rich glycoprotein (HRG) regulates the anticoagulant activity of heparin (Lijnen, 1984, Thromb. Haemost. 51:266). The plasma level of HRG is controlled by factors that include blood type and age (Drasin et al., 1996, Thromb. Res. 84:179), drug treatment (Hennis et al., 1995, Thromb. Haemost. 73:484; Jespersen et al., 1990, Am. J. Obstet. Gynecol. 163:396) and certain disease states (Shigekiyo et al., 2000, Thromb. Haemost. 84:675). Therefore, methods such as those described herein that can be used for enrichment and identification of HRG in a sample are useful for, e.g., diagnostic methods that include assay of HRG.

Vitronectin (VNT) levels are relatively lower in subjects with many types of liver disease, such as alcoholic liver cirrhosis. Vitronectin level is a useful parameter of hepatic synthetic function in patients with liver diseases for example cirrhosis, cancer, viral hepatitis (Inuzuka et al., 1992, Hepatology 15:629; Tomihira, 1991, Fukuoka Igaku Zasshi 82:21; Hogasen et al., 1996, Liver 16:140), and may be diagnostic for progression of such disease Yamada et al., 1997, Res. Commun. Mol. Pathol. Pharmacol. 97:315). Therefore, methods such as those described herein that can be used for enrichment of and identification of VNT are useful for, e.g., diagnostic methods that include assay of HRG.

### Example 6

### Selection of Glycoprotein Standards and an ELISA-Based Screening Assay

To develop an ELISA-based screening assay format for M-LAC, four glycoproteins were selected for study; haptoglobin, α1-antitrypsin, α2-macroglobulin and orosomucoid (α1-acid glycoprotein 1. Specific, affinity-purified chicken IgY antibodies raised against these four proteins (Genway Biotech, San Diego, Calif.) were used in these studies.

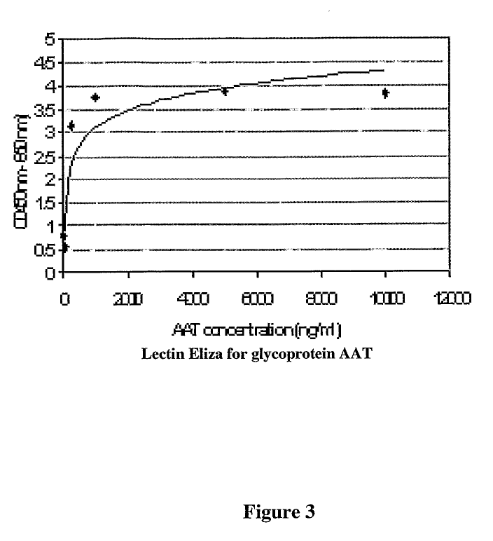

First, the performance of the four IgY antibodies against haptoglobin, a1-antitrypsin, a2-macroglobulin, and orosomucoid was tested. In these experiments, haptoglobin, antitrypsin, a2-macroglobulin, and orosomucoid were diluted in coating buffer and coated onto 96 well plates in a series of concentrations. After blocking with 1% casein in PBS, anti-glycoprotein IgY antibodies, with the exception of anti-α1-antitrypsin, were added at a 1000× dilution in phosphate buffered saline containing 1% Triton® X-100 (PBST). Anti-α1-antitrypsin was used at a 2000× dilution. The antibodies were incubated on the plates and then washed, followed by incubation with HRP-conjugated secondary anti-IgY antibodies (30,000× dilution). The bound antibodies were assayed using a colorimetric reaction by addition of chromogenic substrate 3,3′,5,5′-tetramethylbenzidine (TMB), and the OD₄₅₀ was read. The sensitivity, signal range, and saturation were obtained for the IgY and HRP-conjugated antibodies. This approach can be used to determine optimal parameters for antibody dilutions and assay conditions when developing a M-LAC format using ELISA. A result for the measurement of binding of α1-antitrypsin (AAT) is shown in FIG. 3 as an example.

To study the differential binding to lectins of the four glycoproteins selected to use as standards, a multi-lectin coated plate prepared as described above was used. The 96 well plate was coated with saturating concentrations of individual lectins, each of Jacalin, ConA, or WGA, or a mixture of the three (in equal ratios). The glycoproteins were added to the wells in various concentrations, followed by washing and detection of bound glycoproteins using IgY antibodies, an HRP-conjugated anti-IgY antibody, and substrate reaction. Plates were washed extensively with PBST between each incubation step and all of the steps were performed at room temperature. Each data point of the assay was assayed in duplicate or triplicate, and the experiment was repeated at least five times.

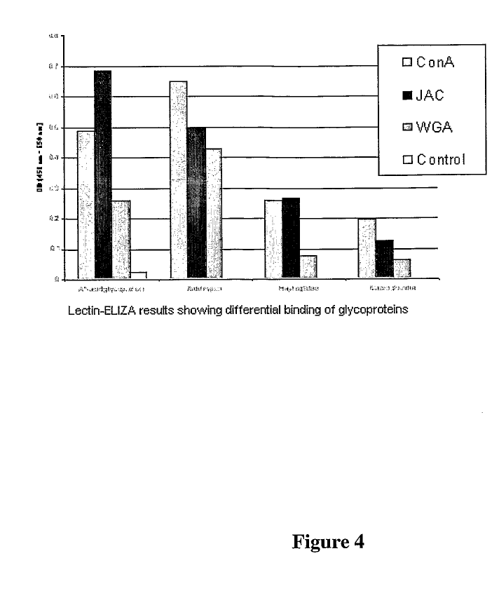

The results of these experiments using multi-lectin ELISA show that glycoproteins bind to lectins with different affinities, as evidenced by OD reading for a specific glycoprotein binding to each of the different lectins. FIG. 4 shows an example of results obtained using the lectin-ELISA format to detect differential binding of four different glycoprotein standards to a lectin array.

The method permits accessing the binding of a specific glycoprotein to a panel of lectins, but also relative quantitation when comparing different lectins. Subtle changes in glycosylation can be detected using this method, e.g., by determining changes in the differential binding to lectins of selected glycoproteins or a panel of glycoproteins. The method can also e used to select optimum binding conditions for a glycoprotein to a panel of lectins. These ELISA-based assays are highly sensitive and can be run with high precision, and therefore are useful for identifying finite changes in glycosylation and furthermore require relatively small amounts of a sample.

To address the reproducibility and specificity of the multi-lectin ELISA, non-specific binding is assessed using multiple controls in which specific steps or reagents are omitted.

The data from multi-lectin ELISA can be correlated with Western blot analysis or data from the lectin column format. Based on the data derived from ELISA, the assay can be miniaturized to an array format on, e.g., a glass slide. Following the similar principles of solid-phase chemistry, a library of lectins can be immobilized onto glass slides directly or through biotinylation.

### Example 7

### Glycoprotein Biomarkers in Serum from Breast Cancer Patients

Some biomarkers of breast cancer are glycoproteins, e.g., Her2 and CEA. Serum samples from subjects who had various stages of breast cancer were analyzed for difference (compared to controls) using M-LAC coupled with nano-LC LTQ MS. The known biomarkers, Her2, BRCA2, carcinoembryonic antigen (CEA), and P53 were identified in a few samples, and the relative levels of these proteins were compared using peak area measurements.

Samples

The serum samples were from five patients diagnosed with invasive breast cancer and serum samples from control subjects (i.e., subjects that did not have diagnosed breast cancer.

Glycoprotein Enrichment Using M-LAC

To isolate glycoproteins from the serum samples, multi-lectin columns were prepared by mixing equal amounts of agarose-bound Con A, agarose-bound WGA, and agarose-bound Jacalin in an empty PD-10 disposable column (GE Healthcare, Piscataway, N.J.). The serum samples (100 μL) were diluted with multi-lectin column equilibrium buffer (20 mM Tris, 0.15 M NaCl, 1 mM MnCl₂ and 1 mM CaCl₂, pH 7.4) to a volume of 1 ml, and were loaded onto newly packed multi-lectin affinity columns. The loaded columns were incubated for 15 minutes at room temperature and then unbound proteins were eluted with 10 ml of equilibrium buffer. The proteins captured on the column were released with 12 ml of displacer solution (20 mM Tris, 0.5 M NaCl, 0.17 M methyl-α-D-mannopyranoside, 0.17 M N-acetyl-glucosamine, and 0.27 M galactose, pH 7.4). The multi-lectin affinity column captured fraction (glycoprotein fraction) was collected and concentrated using 15 ml, 10 kD Amicon filters (Millipore, Billerica, Mass.).

LC-MS/MS

The glycoprotein fractions were digested with trypsin, using the methods described supra. The trypsin-digested peptides were then separated and analyzed on an Ettan MDLC system (GE Healthcare, Piscataway, N.J.) coupled to a LTQ linear ion trap (ThermoElectron, San Jose, Calif.). About 2 μg of each sample was injected onto a Peptide Captrap column (Michrom Bioresources, Inc. Auburn, Calif.) using the autosampler of the MDLC system. To desalt the sample, the trap column was washed with water using 0.1% formic acid at a flow rate of 10 μl/min for four minutes. The flow was directed to the solvent waste through a 10-port valve. After desalting, the valve was switched to direct flow to the separation column. The desalted peptides were then released and separated on a C18 capillary column (packed in-house, Magic C18, 150×0.075 mm). The flow rate was maintained at 400 μl/min and monitored with a flowmeter. The gradient was started at 0% acetonitrile (ACN) with 0.1% formic acid and linearly increased to 35% ACN in 120 minutes, then to 60% ACN in 40 minutes, and to 90% ACN in another 20 minutes, then maintained at 90% ACN for 20 minutes. The Ettan MDLC was operated from UNICORN™ control software (GE Healthcare, Piscataway, N.J.). The resolved peptides were analyzed on an LTQ linear ion trap mass spectrometer with a nano-ESI ion source. The temperature of the ion transfer tube was controlled at 200° C. and the spray voltage was 2.0 kV. The normalized collision energy was set at 35% for MS/MS. Data-dependent ion selection was monitored to select the seven most abundant ions from an MS scan for MS/MS analysis. Dynamic exclusion was continued for a duration of two minutes. Same samples were also analyzed on LC coupled with LTQ FT (ThermoElectron, San Jose, Calif.). The peptides were separated using the same gradient as for the first peptide samples. FT was set for full mass scan and LTQ was set up for data-dependent MS/MS fragmentation.

Bioinformatics

Peptide sequences were identified using the SEQUEST algorithm (Version C1) incorporated into BioWorks, Inc. software (Version 3.1 SR) (ThermoElectron, San Jose, Calif.). Only peptides resulting from tryptic cleavages were searched. The SEQUEST results were filtered by Xcorr vs. charge state. Xcorr was initially used for a match with 1.5 for singly charged ions, 2.0 for doubly charged ions, and 2.5 for triply charged ions. In addition, the SEQUEST results were regenerated using ProteinProphet™ algorithm (University of Washington, Seattle, Wash., Institute for Systems Biology). The identified target proteins were manually checked for the MS/MS spectra from which the peptides were detected for confirmation.

Protein Identification

The glycoprotein samples obtained by enrichment from serum samples using M-LAC columns were trypsinized as described above and the resulting peptides were separated on nano-LC C18 columns, and detected with LTQ MS. Using the SEQUEST algorithm, about 300 proteins were identified in each sample, and total of 1400 non-redundant proteins were found in the total of 10 breast cancer serum samples (the samples were termed BC1 to BC10). The most abundant proteins identified in these glycoprotein-enriched serum samples include alpha-2-macroglobulin, serotransferrin, haptoglobin, hemopexin, alpha-1-antitrypsin, complement C3, apolipoprotein A-I, alpha-1-acid glycoprotein, ceruloplasmin, and complement factor H. Serum albumin was not among the most abundant proteins since it was largely removed by M-LAC with other non-glycosylated proteins.

Identification of Known Biomarkers

In the large amount detected proteins, low level breast cancer biomarkers, Her2, P53, CEA and BRCA2 were found in some of the samples as listed in Table 10. Her2 and BRCA2 peptides were hit by the mass spectrometer detector multiple times (shown as number of hits) in multiple samples. The best identified peptide of the protein was considered as diagnostic peptide candidate for further protein level comparisons. These peptides were detected with good matching with the theoretical MS/MS fragmentations, which is shown as the SEQUEST matching scores listed in Table 10. A filter of SEQUEST scores was set up for the peptide matching as follows, Xcorr≧1.9, 2.2, and 3.75 for +1, +2, and +3 charged ions, respectively; Delta Cn≧0.1; and Rsp≦4.

TABLE 10
Serum Glycosylated Breast Cancer Markers

| Patient Sample | | | | | | | | | | |
|---|---|---|---|---|---|---|---|---|---|---|
| Marker | BC1 | BC2 | BC3 | BC4 | BC5 | BC6 | BC7 | BC8 | BC9 | BC10 |
| ERB2 | | | | | | | + | ++ | + | |
| PLX4 | | | | | | | | + | | + |
| P53 | | | | | | | | +++ | | |
| P73 | ++ | | | | | + | | | | |
| BRC2 | | + | | +++ | | ++ | | | | + |
| MUC2 | + | | | | | | | | | |
| CEA | | | | | | | | + | | |

PLX: PLEXIN A3
MUN: Mucin
Biomarker level: about 50 ng/mL

The protein database search results were also submitted for PeptideProphet analysis using ProteinProphet software (University of Washington, Seattle, Wash., Institute for Systems Biology). Identification of a “prophet” of a peptide using this software indicates the probability of a correct identification. For example, the peptide prophet of the peptide MEEPQSDPSVEPPLSQETFSDLWK (SEQ ID NO:8) of P53 was 94, which meant that the confidence of this identification was 94% and indicated the high possibility of the presence of this peptide in the protein sample digest. The prophet of the diagnostic peptides of BRCA2 and CEA were above or close to 50%, while the Her2 peptide was 31%.

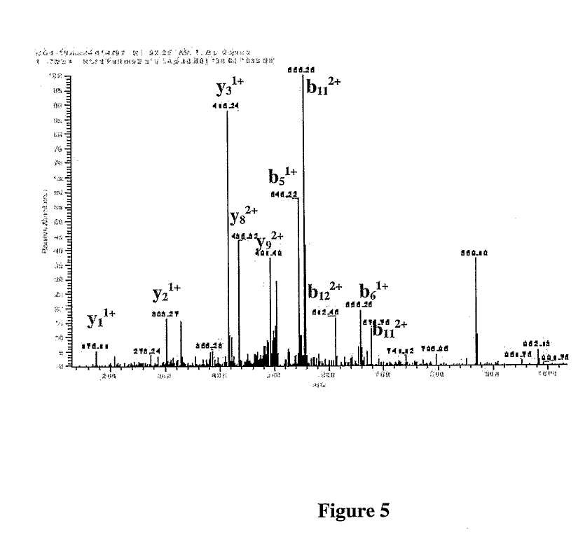

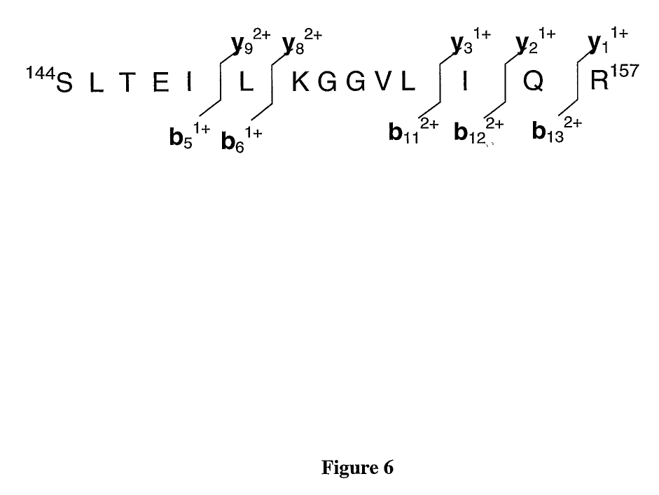

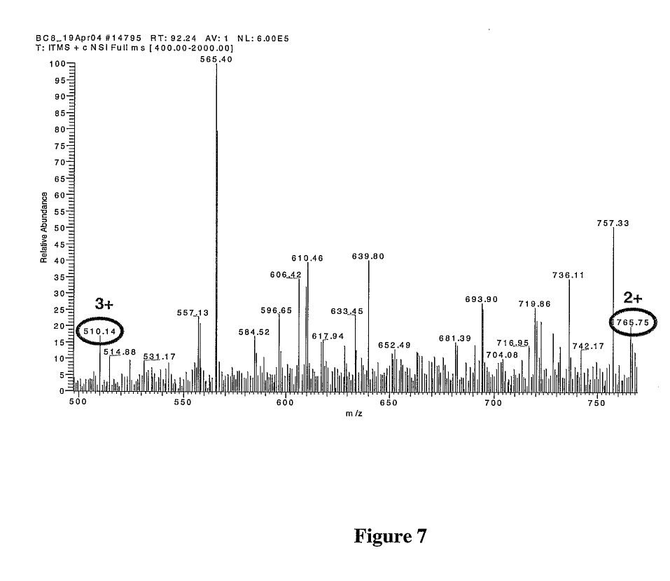

The MS/MS spectrum from which the Her2 peptide, ¹⁴⁴SLTEILKGGVLIQR¹⁵⁷SEQ ID NO:1) was detected was also inspected. This peptide is in the extracellular domain of the protein, and the spectrum is shown in FIG. 5. In this spectrum, extensive peptide fragments were found at a high signal/noise ratio, and most top ions were assigned to expected b and y ions for the peptide. The parent ion of this fragmentation spectrum was a +3 charged ion selected by a data-dependent MS/MS scan. This +3 charge state is due to the two additional amino groups of the basic amino acids, K and R, in addition to the N-terminal amino group. At the acidic condition, these three amino groups are easily protonated. The coordinate charge states were also found in the fragmentations as shown in FIG. 5 and FIG. 6. The y8 and y9 ions include K and R, and are therefore doubly charged, while y1, y2 and y3 only have R in their sequence and are singly charged. The same phenomena were shown for the b ions. The b5 and b6 ions only are singly charged due to an N-terminal amine, while b11, b12, and b13 ions are doubly charged since they include K as well as the N-terminal amine. In these fragmentations, the assigned y and b ions were paired with the y3 and b11 ions that gave the most intense signals. The cleavages between large hydrophobic residues, such as between leucine and isoleucine, are favored in this fragmentation, because the weak inductive effect of the alkyl residues (electron donating) helps stabilize the charge. In addition, the doubly charged peptide ion was also found in the same full MS scan, although the signal is closer to the noise level, as shown in FIG. 7. Based on the MS/MS spectrum investigation, the possibility of a false positive identification of this particular peptide from Her2 was eliminated. Using the same methods, all of the MS/MS spectra of the diagnostic peptides were examined and confirmed.

These diagnostic peptides were identified in some of the breast cancer samples as listed in Table 11, but not all of the peptides were identified in all samples. However, the non-identification of the peptide did not necessarily mean that the protein was not in the sample.

TABLE 11
Identified Known Breast Cancer Biomarkers and Detected Peptides

| | | |
|---|---|---|
| | | Number of patient samples |
| | Total | in which the proteins were |
| Patient | Number of | found |

| groups | patients | Her2^{a} | P53 | CEA | BRC2 |
|---|---|---|---|---|---|
| Control | 5 | 0 | 3 | 0 | 0 |
| DCIS | 5 | 4 | 5 | 1 | 1 |
| Invasive | 5 | 5 | 5 | 4 | 3 |

^{a}The identification of Her2 was confirmed using ELISA with the range of plasma concentration of 8.5 ng/ml to 24.4 ng/ml in the 10 breast cancer patient samples.

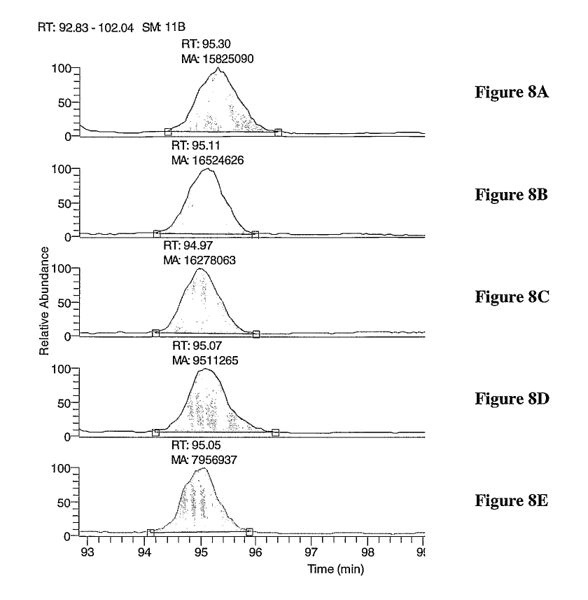

The mass spectrometer could not be used to identify the peptide ion when selecting ions to generate MS/MS spectra using data-dependent mode, especially for the low level peptides. To avoid possible false negative results, the peak areas of selected peptides were compared for quantification. Specifically, the extracted ion chromatogram peaks were selected if they were within 1 amu of the expected mass, +/−0.5 minutes of the observed retention time, and above a S/N (signal to noise) of 5:1. The criteria were determined by verifying that the standard deviation for retention time over multiple runs was 0.5 minutes. FIG. 8 shows the examples of extracted ion chromatograms of the peptide of ceruloplasmin, DLYSGLIGPLIVCR (SEQ ID NO:2) (M/Z signal 788.6) from the M-LAC enriched breast cancer patient serum samples. The peak areas of the peptide ions were extracted from the complex chromatograms. FIG. 7 shows the examples of extracted ion chromatograms (EIC) of the peptide of Her2, ¹⁴⁴SLTEILKGGVLIQR¹⁵⁷ (SEQ ID NO:1) (M/Z signal 509.9) from the 15 samples (controls and patients). The peak area was integrated at the retention time where the peptide was detected, or the close retention time if the peptide was not detected in the sample. Other than BC7, BC8, and BC9, from which the peptide, ¹⁴⁴SLTEILKGGVLIQR¹⁵⁷ (SEQ ID NO:1) was detected, a significant peak with signal/noise greater than 5 was able to be integrated out in each of the samples BC1, BC2, BC4, BC5, BC6 and BC10. The appearance of these peaks also indicated the presence of the peptide in these samples. However, the selection of a data-dependent scan in the LC-MS/MS analysis missed the selection of these ions since this ion is present in low levels and the peak is small. The lack of detection of this peptide could also be due to the spectra not being clear enough to be sequenced with acceptable identification scores by SEQUEST algorithm. Even so, the retention times of these low score spectrums were used for positioning the peak in EIC.

Biomarker Levels in Breast Cancer

The peak areas of the peptide ions of four biomarkers are listed in Table 12.

TABLE 12
Peak areas of detected peptide ions of breast cancer biomarkers
in extracted ion chromatograms of control and breast cancer
patient samples

| Sample | Breast cancer stage | ERB2 | CEA | P53 | BRC2 |
|---|---|---|---|---|---|
| Control 1 | Cancer free | N/A | N/A | N/A | N/A |
| Control 2 | Cancer free | N/A | N/A | 0.2 | N/A |
| Control 3 | Cancer free | N/A | N/A | 0.2 | N/A |
| Control 4 | Cancer free | N/A | N/A | N/A | N/A |
| Control 5 | Cancer free | N/A | N/A | 0.1 | N/A |
| BC 1 | DCIS | 1.3 | 0.28 | 0.3 | N/A |
| BC 2 | DCIS | 0.2 | N/A | 0.2 | N/A |
| BC 3 | extensive DCIS | N/A | N/A | 0.2 | N/A |
| BC 4 | DCIS | 0.5 | N/A | 0.6 | 1.1 |
| BC 5 | DCIS (intermediate nuclear | 0.8 | N/A | 2.3 | N/A |
| | grade) | | | | |
| BC 6 | Invasive carcinoma & | 1.0 | N/A | 2.9 | 0.7 |
| | DCIS II/III | | | | |
| BC 7 | Metastatic carcinoma | 3.0 | 0.5 | 0.6 | N/A |
| | (invasive lobular carcinoma | | | | |
| | in situ) grade II/III | | | | |
| BC 8 | Invasive carcinoma (no | 1.8 | 0.8 | 2.0 | 0.2 |
| | DCIS identified) T4 | | | | |
| | pNxMx | | | | |
| BC 9 | Metastatic adenocarcinoma | 2.0 | 0.2 | 1.7 | N/A |
| | T1bpN1aMx | | | | |
| BC 10 | Metastatic breast cancer; | 0.9 | 1.7 | 7.8 | 4.9 |
| | Invasive ductal carcinoma | | | | |
| | Grade II/III | | | | |

ERB2: ELTEILKGGVLIQR M/Z 509.9
CEA: EVLLLVHNLPQDPR M/Z 823.1
BRC2: LAAMECAFPKEFANR M/Z 879.1
P53: MEEPQSDPSVEPPLSQETFSDLWK M/Z 926.3

The Her2 peptide peak was found in most of the ten breast cancer samples. The peak areas in the invasive cancer samples were relatively larger and ranged form 0.89 to 3.04×10⁶ counts. The peak areas in ductal carcinoma in situ (DCIS) samples were less than 1.0×10⁶ counts except for BC1. BC1 also had higher level of CEA with the peak area of 0.28×10⁶ counts, while CEA peptide peak was not found in the other four DCIS samples. From the four out of five invasive breast cancer samples, CEA was detected with peak area from 0.23 to 1.67×10⁶ counts. Her2 and CEA serum levels have been found to be useful marker to monitor the stage of breast cancer. The relative levels of these two markers in the DCIS and invasive breast cancer patients identified using the methods described herein also showed good agreement with the stage of the cancer.

The P53 peptide peak was found in all the ten breast cancer patient serum samples, but the peak areas integrated out from the invasive stage samples were relatively higher. The peak of P53 peptide was also found in most of the samples including controls with the area similar to those in the DCIS samples.

BRC2 peptide peak was found in some of the breast cancer samples (DCIS and invasive) but not in the controls. There was no significant correlation between the peak area and the stage of the breast cancer.

### Example 8

### Detection of Silent Changes in Glycosylation

To demonstrate the utility of the methods described herein for evaluating changes in glycosylation patterns, a multi-lectin column containing Con A, WGA, and Jacalin lectin was used to screen glycoproteins with known glycosylation pattern changes. The presence of a glycoprotein in a given displacer fraction was determined by LC-MS/MS analysis of a tryptic digest. Neuraminidase was used to modify the oligosaccharide chains present in human transferrin, and neuraminidase and fucosidase were used to modify glycoproteins present in human serum. Then, by comparison with the untreated samples, a distribution shift of the enzyme-treated serum glycoproteins in the displacement fractions isolated from the multi-lectin column. The fractions were analyzed using a protein assay, SEQUEST rank comparison, and peak area measurement from the extracted ion chromatogram. The results indicated that the multi-lectin affinity column (M-LAC) is sensitive to changes in the content of sialic acid and fucosyl residues present in serum glycoproteins, and can has the potential to be used to screen serum proteins for glycosylation changes due to disease. In addition, the use of a glycosidase to induce specific structural changes in glycoproteins can support the development of multi-lectin column formats specific for detecting changes in the glycoproteome of certain diagnostic fluids and types of disease.

Materials

Agarose-bound Con A with a protein concentration of 6 mg lectin/ml gel and a binding capacity of more than 4 mg ovalbumin/ml gel, agarose-bound WGA with a protein concentration of 7 mg lectin/ml gel and a binding capacity of 8 mg NGA/ml gel and agarose-bound Jacalin with a protein concentration of 4 mg lectin/ml gel and a binding capacity of more than 4 mg monomeric IgA/ml gel were obtained from Vector Laboratories (Burlingame, Calif.). Trypsin (sequence grade) was purchased from Promega (Madison, Wis.). Human serum, bovine serum albumin (BSA), transferrin, α(2→3,6,8,9) neuraminidase Arthrobacter ureafaciens (proteomics grade, EC number: 3.2.1.18) and α-1,6-fucosidase solution (recombinant) were purchased from Sigma-Aldrich (St. Louis, Mass.). Novex pH 3-10 IEF gel and SimpleBlue™ SafeStain were purchased from Invitrogen (Philadelphia, Pa.). Coomassie Plus™ protein assay reagent was purchased from Pierce (Rockford, Ill.).

Treatment of Transferrin with Neuraminidase.

Human transferrin at a concentration of 10 mg/ml was dissolved in a 100 mM phosphate buffer at pH 7.4. A sample of transferrin solution (30 μL) was diluted using reaction buffer (5×), then incubated with four units of neuraminidase at 37° C. for three hours (one unit enzyme releases 1 nmol of 4-methylumbelliferone from 2-(4-methylumbelliferyl) α-D-N-acetylneuraminic acid per minute at pH 5.5 at 37° C.). The sample, asialotransferrin, was immediately chromatographed on the M-LAC column.

Treatment of Human Serum with Neuraminidase or Fucosidase

Human serum (100 μL) was incubated with 25 units of neuraminidase in the reaction buffer (pH 5.5) at 37° C. overnight or a serum sample was incubated with 0.038 unit of fucosidase in the reaction buffer (pH 5.0) at 37° C. for 12 hours (one unit enzyme will release 1.0 μmol of methylumbelliferone from 4-methylumbelliferyl-α-L-fucoside per min at pH 5.0 at 37° C.).

Sequential Fractionation of Glycoproteins Using Three Displacers

A multi-lectin column was prepared by mixing 0.5 ml of agarose-bound Con A, 0.5 ml of agarose bound WGA, and 0.5 ml of agarose bound Jacalin lectin in an empty PD-10 disposable column. In a series of experiments the following samples: transferrin (300 μg), asialotransferrin (300 μg), untreated serum (100 μL), neuraminidase-treated serum (100 μL), and fucosidase-treated serum (100 μL) were each diluted with M-LAC equilibration buffer (20 mM Tris, 0.15 M NaCl, 1 mM MnCl₂ and 1 mM CaCl₂, pH 7.4) to a volume of 1 ml, and then loaded individually onto separate M-LAC columns. To avoid cross contamination, separate multi-lectin columns were used for each study. After a 15 minute incubation, the unbound proteins were eluted with 10 ml of equilibration buffer. To fractionate the captured proteins based on glycosylation motifs, the sample was eluted with the displacers as follows. Proteins bound to Jacalin lectin were first released with 4 ml of 0.8 M galactose in 20 mM Tris buffer (pH 7.4) containing 0.5 M NaCl. Then Con A-selected proteins were released with 4 ml of 0.5 M methyl-α-D-mannopyranoside in a 20 mM Tris buffer (pH 7.4) containing 0.5 M NaCl. Finally, the WGA selected proteins were released with 4 ml of 0.5 M N-acetyl-glucosamine in 20 mM Tris buffer, pH 7.4 containing 0.5 M NaCl. The three fractions were concentrated with a 10 kD Amicon filter (Millipore, Billerica, Mass.). All studies were performed in duplicate to test reproducibility of the enzymatic digestion.

Protein Assay

The amount of protein in M-LAC the displacement fractions of transferrin and asialotransferrin were measured using Bradford protein assay. BSA solutions in a series of concentrations (200 mg/ml, 400 mg/ml, 600 mg/ml, 800 mg/ml, and 1000 μg/ml, were used as standards. Standards and samples (10 μL each) were pipetted into a 96 well flat bottom plate (Corning Inc. Corning, N.Y.), and 300 μL Coomassie Plus™ protein assay reagent was added. After a ten minute incubation at ambient temperature, the samples were measured with a UV detector at a wavelength of 595 nm using a SPECTRA max plate-reader (Molecular Devices, Sunnyvale, Calif.).

IEF Separation

Transferrin, asialotransferrin, and the corresponding displacement fractions collected from the M-LAC were loaded (5 μg protein per sample) onto a Novex IEF gel (1.0 mm, 10 well, pH 3-10). The proteins were focused on the gel with a voltage gradient. The voltage was first kept at 100 V for 1 hour, changed to 200 V for another hour, and then finished at 500 V (30 minutes). The gel was then stained using SimpleBlue™ SafeStain. The samples were analyzed in the absence of detergent and reducing agents.

LC-MS/MS

The glycoprotein fractions were digested with trypsin, using a procedure described previously as described in the art and herein. The trypsin-digested peptides were then separated and analyzed on an Ettan MDLC system (GE Healthcare, Piscataway, N.J.) coupled to a LTQ linear ion trap (ThermoElectron, San Jose, Calif.). About 2 μg of each sample was injected onto a Peptide Captrap column (Michrom Bioresources, Inc., Auburn, Calif.) using the autosampler of the MDLC system. To desalt the sample, the trap column was washed with water containing 0.1% formic acid at a flow rate of 10 μL/min for four minutes. The flow was directed to the solvent waste through a 10-port valve. After desalting, the valve was switched to directing flow to the separation column. The desalted peptides were then released and separated on a C18 capillary column (packed in-house, Magic C18, 150×0.075 mm). The flow rate was maintained at 400 μL/min monitored with a flow-meter. The gradient was started at 0% acetonitrile (ACN) with 0.1% formic acid and linearly increased to 35% ACN over 60 minutes, then to 60% ACN in 15 minutes, and to 90% ACN in another 5 minutes, then kept at 90% ACN for 10 minutes. An Ettan MDLC was operated from UNICORN™ control software (GE Healthcare, Piscataway, N.J.). The resolved peptides were analyzed on an LTQ linear ion trap mass spectrometer with a nano-ESI ion source. The temperature of the ion transfer tube was controlled at 200° C. and the spray voltage was 2.0 kV. The normalized collision energy was set at 35% for MS/MS. Data-dependent ion selection was monitored to select the most abundant seven ions from a MS scan for MS/MS analysis. Dynamic exclusion was continued for duration of three minutes.

Bioinformatics

Peptide sequences were identified using SEQUEST algorithm (Version C1) incorporated in BioWorks software (Version 3.1 SR) (ThermoElectron, San Jose, Calif.). Only peptides resulting from tryptic cleavages were searched. The SEQUEST results were filtered by Xcorr vs. charge state. Xcorr was used for a match with 1.5 for singly charged ions, 2.0 for doubly charged ions, and 2.5 for triply charged ions. The proteins with more than one peptide detected were considered.

Distribution of Transferrin and Asialotransferrin in Displacement Fractions

As described above, transferrin was treated with neuraminidase to generate asialotransferrin. The treated sample was loaded on an M-LAC column and an equal amount of an untreated transferrin sample was loaded onto a separate multi-lectin column. Unbound proteins were captured in the flow-through fraction and the bound proteins were collected in separate fractions after elution by each displacer.

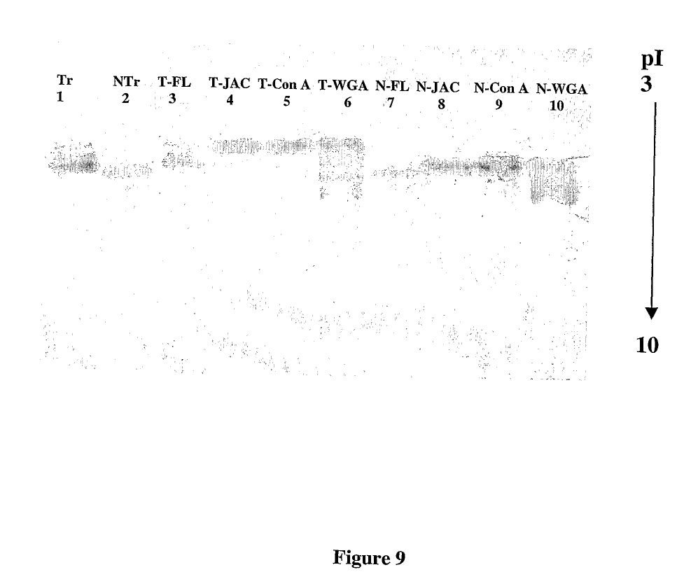

The flow-through portion of the transferrin sample, as expected, contained mainly the non-glycosylated fraction of the sample. FIG. 9 illustrates an IEF separation of the fractions, including intact transferrin and asialotransferrin. The higher pI of the components in the asialotransferrin relative to the untreated sample indicated the successful release of sialic acid residues from the glycan structures of transferrin by neuraminidase. With equal amounts of proteins loaded, the WGA fraction showed multiple bands, which indicated that sialic acid variants were present in low levels in the transferrin sample that was bound by WGA.

Protein concentrations were assayed for each collected fraction, which demonstrated similar recoveries between the intact protein and asialo protein after M-LAC separation, 86% and 85% respectively. These are typical results for this procedure and indicate good performance. Compared with the M-LAC results for transferrin, the amount of asialotransferrin captured by WGA was reduced by about 40%, while the amount of protein captured by Jacalin and Con A lectin were correspondingly increased about 6% and 13%. This distribution shift was reproducible, and was attributed to a decreased affinity of the asialo protein for WGA, which has a specific affinity to sialic acid. Also, in the glycan structures of transferrin, more galactosyl residues are exposed after removal of sialic acid residues, which increased the affinity of this protein to the Jacalin lectin. In a similar manner, Con A exhibited increased affinity to asialotransferrin compared to WGA.

Distribution Shift of Serum Glycoproteins after Neuraminidase Treatment

Human serum was treated with neuraminidase to cleave sialic acid residues from the glycan forms present in serum glycoproteins, and the product was loaded on a M-LAC column as described above. The glycoproteins captured by each lectin immobilized in the M-LAC column were sequentially released with the three specific lectin displacers. After trypsin digestion, each fraction was analyzed by LC-MS/MS, and the proteins were identified using the SEQUEST algorithm. To investigate the relative abundance change of the glycoproteins in each lectin fraction, the SEQUEST ranks of the lectin fractions before and after enzyme treatment were compared. This parameter represents the quality of assignment of a protein in a sample. While SEQUEST ranking (a smaller the number generally indicates a higher the concentration) is not a direct measure of protein concentration, it can be used to describe trends in changes of relative protein concentration due to dependence of the algorithm on parameters, such as number of peptides detected and quality of spectra in a given peptide identification. The average SEQUEST rank change, which represents the summation of protein level changes (in terms of absolute magnitude) between the two samples, is listed in Table 13.

TABLE 13
Average SEQUEST rank^{a} shift of serum glycoproteins in lectin
displacement fractions from M-LAC after enzymatic treatment

| Average rank shift^{b} | Neu. Serum^{c} | Fuc. Serum^{d} |
|---|---|---|
| Jacalin fraction | 7 | 9 |
| Con A fraction | 18 | 6 |
| WGA fraction | 31 | 18 |

^{a}The SEQUEST rank represents the probability of assignment of a given protein.
^{b}The average of the SEQUEST rank differences of proteins between an enzyme-treated sample and the intact serum sample in each lectin displacement fraction. The average of SEQUEST rank differences between duplicate analyses of untreated serum samples was used as a control.
^{c}Neuraminidase-treated serum.
^{d}Fucosidase-treated serum.

The average of the SEQUEST rank differences between duplicate serum samples was used as a control. Compared with the control value, the neuraminidase-treated serum sample had an obvious SEQUEST rank shift in all three displacer fraction samples, i.e., 7, 18, and 31 for the Jacalin, Con A, and WGA fractions respectively (Table 13). This result indicated that neuraminidase digestion did cause a shift in the distribution of the proteins in the three displacer fractions. These data also demonstrated that the multi-lectin column is sensitive to the changes of sialic acid content in the glycan forms.

In addition, the distribution of five different glycoproteins were investigated in the human serum samples, with and without neuraminidase treatment. These proteins, transferrin, haptoglobin, alpha-2HS-glycoprotein, beta-2-glycoprotein, and alpha-1-acid glycoprotein, have been reported as containing sialic acid residues in their carbohydrate moieties (Gambino et al., 1997, J. Lipid Mediat. Cell Signal. 17:191-205; Schousboe, 1983, Int. J. Biochem. 15:35-44; Watzlawick et al., 1992, Biochemistry 31:12198-12203). Some of these structures have shown a correlation to diseases in terms of sialic acid-related glycosylation pattern changes (Inoue et al., 1999, Electrophoresis 20:452-457);

Wang et al., 1993, Alcohol Alcohol Suppl. 1A:21-28; Bradley et al., 1977, Cancer 40:2264-2272). The SEQUEST rank of the selected glycoproteins from untreated serum and neuraminidase-treated serum were compared for each displacement fraction (Table 14). According to the SEQUEST rank comparison, the abundance of these proteins were reduced in the WGA fraction after neuraminidase treatment, although some trends were more substantial than the others. In the case of transferrin, alpha-2HS-glycoprotein and beta-2-glycoprotein, the proteins could not be identified at all in the WGA fraction after neuraminidase digestion. Instead, the levels of transferrin and alpha-2HS-glycoprotein were raised in the Jacalin fraction.

TABLE 14
SEQUEST rank of selected glycoproteins in M-LAC displacer fractions
from serum and neuraminidase-treated serum samples

| | Sample | | | | | |
|---|---|---|---|---|---|---|
| | Human serum | | | Neu.-treated serum | | |
| Displacer fraction | JAC | Con A | WGA | JAC | Con A | WGA |
| Transferrin | 2 | 2 | 19 | 1 | 2 | X^{a} |
| Haptoglobin | 9 | 7 | 4 | 9 | 7 | 2 |
| Alpha-2HS-glycoprotein | 14 | 32 | 81 | 7 | 31 | X |
| Beta-2-glycoprotein | 20 | 20 | 52 | 23 | 24 | X |
| Alpha-1-acid- | 21 | 33 | 7 | 27 | 96 | 23 |
| glycoprotein | | | | | | |

^{a}The protein was not identified in the sample. In the case of the WGA fraction from transferrin and alpha-2HS-glycoprotein, the peptide could be identified by extracted ion monitoring.

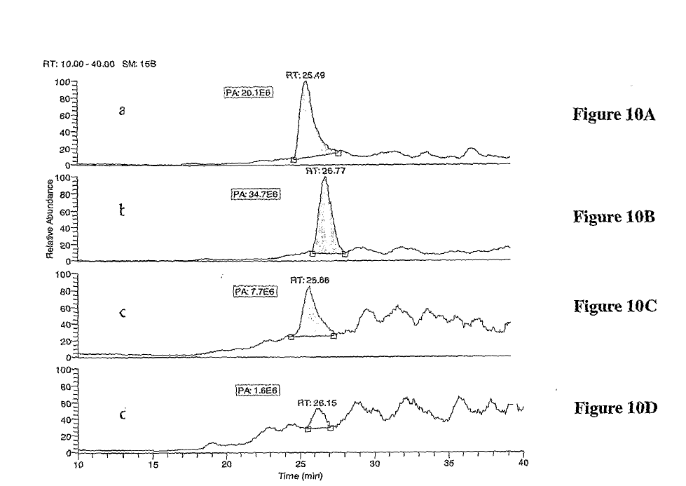

The SEQUEST rank is a semi-quantitative parameter for evaluating the relative abundance of a protein in a complex sample. Therefore, to further investigate the distribution shift of the selected set of proteins between the displacer fractions, peak areas of selected peptides for quantification were evaluated. Specifically, the extracted ion chromatogram peaks were selected if they were within 1 amu of the expected mass, +/−0.5 minutes of the observed retention time, and above a S/N (signal to noise) of 5:1. This criterion by arrived at by verifying that the standard deviation for retention time over multiple runs was 0.5 min. FIG. 10 shows the examples of extracted ion chromatograms of the peptide of transferrin, KPVEETANCHLAR (SEQ ID NO:3) (M/Z signal 530.2) from the M-LAC displacer fractions of serum and neuraminidase-treated serum samples. Within the acceptable retention time window, a small peak area of 1.6 (E6 counts) was integrated from the WGA fraction of the neuraminidase-treated serum sample (FIG. 10D).

A comparison of the peak areas demonstrates that the distribution of transferrin in serum that was treated with neuraminidase shifted from the WGA fraction to the Jacalin and Con A fractions. This result is consistent with both the SEQUEST rank comparison and the study transferrin standards (supra). This result was confirmed by the measurement of peak areas of a second selected transferrin peptide, FDEFFSEGCAPGSK (SEQ ID NO:9), (Table 15). Another comparison of peak areas (Table 15) indicated that haptoglobin exhibited a similar shift from the WGA fraction to the Jacalin and Con A fractions after the serum sample was treated with neuraminidase. The distribution changes observed for beta-2-glycoprotein and alpha-1-acid glycoprotein in Jacalin and Con A fractions were similar to the other two proteins but could only be detected by peak area measurement and not using SEQUEST rank comparisons. For each protein, multiple peptides, if detected, were selected for peak area measurements, and the peak area comparison between different runs showed the similar trend as shown in Table 15.

TABLE 15

Selected peptide peak areas (x E6 counts) of targeted
glycoproteins identified in M-LAC displacer fractions
from serum and neuraminidase-treated serum samples

| | | |
|---|---|---|
| | | Neu.-treated |
| M/Z | Human serum | serum |

| | | | | | | | | |
|---|---|---|---|---|---|---|---|---|
| Protein | Selected peptide ^{a} | Signal ^{b} | JAC | Con A | WGA | JAC | Con A | WGA |

| | | | | | | | | |
|---|---|---|---|---|---|---|---|---|
| Transferrin | KPVEEYANCHLAR | 530.2 | 19 | 20 | 7.7 | 24 | 35 | 1.6 |
| | (SEQ ID NO:3) | | | | | | | |
| Transferrin | FDEFFSEGCAPGSK | 790.1 | 7.0 | 7.8 | 5.0 | 8.3 | 11 | 0 |
| | (SEQ ID NO:9) | | | | | | | |
| Haptoglobin | YVMLPVADQDQCIR | 854.6 | 10 | 39 | 236 | 13 | 47 | 163 |
| | (SEQ ID NO:4) | | | | | | | |
| Alpha-2HS- | AQLVPLPPSTYVEFTVSGTDCVAK | 1290.32 | 4 | 2.6 | 1.7 | 7.4 | 2.6 | 0.9 |
| glycoprotein | (SEQ ID NO:5) | | | | | | | |
| Beta-2- | ATVVYQGER | 512.3 | 2.6 | 3.5 | 1.8 | 3.8 | 8.4 | 0 |
| glycoprotein | (SEQ ID NO:6) | | | | | | | |
| Alpha-1-acid- | EQLGEFYEALDCLR | 872.3 | 9.7 | 9.2 | 129 | 12 | 11 | 0 |
| glycoprotein | (SEQ ID NO:7) | | | | | | | |

^{a} The selected peptide was identified by data-dependent MS/MS in LTQ, and the peak area of the extracted ion chromatogram was measured at expected retention time (±0.5 min).
^{b} The peak area of a selected peptide of the protein was extracted at the M/Z signal where the peptide was detected.

These results demonstrate that the M-LAC approach, with the sequential use of displacers can be used to detect shifts in glycoprotein distributions in a complex sample. This confirms this approach for use in identification of biomarkers that have different glycosylation patterns that are related to disease status.

Distribution Shift of Serum Glycoproteins after Fucosidase Treatment

Changes in the level of fucosylation have been shown to occur in a significant number of diseases. Studies of fucosyltransferases have suggested important changes in fucose metabolism in cancer (Thompson et al., 1992) Cancer Lett. 65:115-121) and abnormally-fucosylated haptoglobin was found to be elevated in serum in patients with active rheumatoid arthritis (Thompson et al., Clin. Chim. Acta 184:251-258) and carcinoma of the ovary and breast (Thompson et al., 1992, supra). The level of fucosylation of alpha-1-acid glycoprotein is significantly higher in patients with liver disease (Ryden et al., 2002, Clin. Chem. 48:2195-2201).

To determine whether M-LAC can be used for detecting such changes, human serum was treated with a fucosidase to release fucosyl residues from oligosaccharides present in glycoproteins, and then fractionated on an M-LAC column with a sequential use of displacers specific for each lectin. The changes in the SEQUEST rank of the displacement fractions of the enzyme-treated sample relative to the original serum sample were shown in Table 13. These changes indicated that the glycosylation pattern change induced by fucosidase digestion resulted in a distribution shift between the three displacement fractions. Although none of the three lectins in the M-LAC column have an absolute affinity to fucose, the loss of fucosyl residues from the glycan influences the environment of other sugar residues (e.g., N-acetylglucosamine), which also have an affinity to the lectins (e.g., WGA). These results indicate that the M-LAC column containing Jacalin, Con A, and WGA can detect a glycosylation pattern change as a result of the gain or loss of fucosyl residues in the glycan forms. Another implication of this study is that broader specificity of lectins compared to antibodies are better suited to detect subtle structural changes in glycosylation motifs.

The distribution shift detected using M-LAC in the glycoproteins caused by fucosidase digestion was not as significant as neuraminidase digestion. There are several possible explanations for this including the properties of a specific lectin, such as WGA, which has affinity for sialic acid. Also, sialic acid is more common than fucosyl residues in glycan moieties and is charged at physiological pH values. Thus, the loss of sialic acid in the samples resulted in a more dramatic change in distribution between the three displacer fractions. Therefore, by optimizing the combination of lectins immobilized in M-LAC, such as including a combination of lectins with specificity to fucose, M-LAC will be more sensitive to glycosylation changes at fucosyl residues.

Although the distribution shift induced by the use of fucosidase was not as great in terms of the average SEQUEST rank change of the serum glycoproteins, some individual fucosylated glycoproteins showed a detectable distribution shift. For example, according to the SEQUEST rank and the peak area comparisons (Table 16 and Table 17), after the treatment of fucosidase, a significant fraction of haptoglobin and alpha-1-acid-glycoprotein glycoforms shifted from the WGA fraction to the Jacalin fraction, and some haptoglobin shifted to the Con A fraction.

TABLE 16
SEQUEST rank of Selected Glycoproteins Identified in Displacer
Fractions from Serum and Fucosidase-Treated Serum Samples

| | Sample | | | | | |
|---|---|---|---|---|---|---|
| | Human serum | | | Fuc.-treated serum | | |
| Displacer fraction | JAC | Con A | WGA | JAC | Con A | WGA |
| Haptoglobin | 9 | 7 | 4 | 7 | 5 | 2 |
| Alpha-1-acid- | 21 | 33 | 7 | 19 | 38 | 9 |
| glycoprotein | | | | | | |
| Apo A | 4 | 19 | 10 | 5 | 12 | 24 |
| Ceruloplasmin | 24 | 14 | 8 | 6 | 8 | 28 |
| Inter-alpha-trypsin | 6 | 22 | 9 | 31 | 13 | 30 |
| inhibitor | | | | | | |
| Pregnancy zone protein | x | 34 | 7 | 6 | 25 | 17 |

TABLE 17
Selected Peptide Peak Areas (xE6 counts) of Glycoproteins Identified
in M-LAC Displacer Fractions from Serum and Fucosidase-Treated Serum
Samples

| | | M/Z | Human serum | | | Fuc.-treated serum | | |
|---|---|---|---|---|---|---|---|---|
| Protein | Selected peptide^{a} | Signal^{b} | JAC | Con A | WGA | JAC | Con A | WGA |
| Haptoglobin | YVMLPVADQDQCIR | 854.6 | 10 | 40 | 236 | 22 | 51 | 82 |
| Alpha-1-acid- | EQLGEFYEALDCLR | 872.3 | 9.7 | 9.2 | 129 | 17 | 10 | 50 |
| glycoprotein | | | | | | | | |
| Apolipoprotein A | VSFLSALEEYTK | 694.0 | 40 | 12 | 60 | 43 | 12 | 16 |
| Ceruloplasmin | MFTTAPDQVDKEDEDFQESNK | 826.0 | 2.0 | 7.8 | 9.1 | 6.2 | 9.8 | 11 |
| Inter-alpha- | IYGNQDTSSQLK | 678.5 | 0.56 | 0.5 | 0.74 | 1.5 | 0 | 0 |
| trypsin inhibitor | | | | | | | | |
| Pregnancy | ATVVYQGER | 512.3 | 0 | 6.6 | 11 | 0 | 16 | 5.2 |
| Zone protein | | | | | | | | |

^{a}The selected peptide was identified by data dependent MS/MS in LTQ, and the peak area of the extracted ion chromatogram was measured at expected retention time (±0.5 min).
^{b}The peak area of a selected peptide of the protein was extracted at the M/Z signal where the peptide was detected.

The distribution of ceruloplasmin, in which variable fucosylation was shown to be a cause of micro-heterogeneity observed in ceruloplasmin samples (Kolberg et al., 1983, Hoppe Seylers Z. Physiol. Chem. 364:111-117), was shifted to the Jacalin fraction after fucosidase treatment. In addition, other glycoproteins, such as apolipoprotein A-I, inter-alpha-trypsin inhibitor, and pregnancy zone glycoprotein, also had altered distribution between the three displacement fractions after the treatment with fucosidase (Table 16 and Table 17). The amount of apolipoprotein A-I observed in the WGA displacement fraction decreased, which was shown in both Sequest rank comparison and selected peptide peak area comparison. Inter-alpha-trypsin inhibitor and pregnancy zone protein also had a reduction in the WGA displacement fraction after treatment with fucosidase, with a corresponding increase in the Con A fraction. These results indicate that a change in fucosylation in a sample can be detected by a change in the distribution of the glycoproteins in the three displacement fractions from an M-LAC study. Such analysis can be a valuable tool to screen global basis for subtle changes in the glycosylation of biomarkers in diseases, such as cancer and liver diseases.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising at least three different lectins attached to at least one solid support.

2. The composition of claim 1, wherein at least four lectins are attached to the solid support.

3. The composition of claim 1, wherein at least five lectins are attached to the solid support.

4. The composition of claim 1, wherein the solid support comprises at least one bead.

5. The composition of claim 3, wherein the composition comprises at least two beads and one type of lectin is attached to each bead.

6. The composition of claim 1, wherein the solid support comprises a gel.

7. The composition of claim 1, wherein the solid support comprises agarose.

8. The composition of claim 1, wherein the solid support is in a column.

9. The composition of claim 1, wherein the solid support is a microtiter plate.

10. The composition of claim 1 wherein the lectins are selected from the group consisting of concanavalin A (Con A), wheat germ agglutinin (WGA), Jacalin, lentil lectin (LCA), and peanut lectin (PNA).

11. The composition of claim 1, wherein the lectins are selected from the group consisting of Lens culinaris agglutinin (LCA), Griffonia (Bandeiraea) simplicifolia lectin II (GSLII) Aleuria aurantia Lectin (AAL), Hippeastrum hybrid lectin (HHL, AL), Sambucus nigra lectin (SNA, EBL), Maackia amurensis lectin II (MAL II), Ulex europaeus agglutinin I (UEA I), Lotus tetragonolobus lectin (LTL), Galanthus nivalis lectin (GNL), Euonymus europaeus lectin (EEL), and Ricinus communis agglutinin I (RCA).

12. The composition of claim 1, wherein the lectins are present in equal ratios in the composition.

13. A method of isolating glycoproteins from a sample, the method comprising:
a. contacting the composition of claim 1 with a sample under conditions that promote binding of glycoproteins in the sample to the lectins, thereby providing a bound sample;
b. removing an unbound sample from the contacted composition; and
c. eluting the glycoproteins from the bound sample.

14. The method of claim 13, wherein the composition comprises at least three, four, or five different lectins.

15. The method of claim 13, wherein the sample is a biological fluid, a tissue preparation, of a cell culture preparation.

16. The method of claim 15, wherein the biological fluid sample is plasma, serum, blood, urine, lacrimal secretion, seminal fluid, vaginal secretion, sweat, or cerebrospinal fluid.

17. The method of claim 13, wherein at least two different elution steps are performed.

18. A method of isolating a glycoprotein biomarker, the method comprising:
a. contacting a composition, comprising at least three different lectins attached to a solid support, with a sample containing the biomarker, under conditions that promote binding of glycoproteins in the sample to the lectins, thereby providing a bound sample;
b. removing unbound sample from the contacted composition; and
c. eluting at least one glycoprotein from the bound sample, wherein the biomarker is in the eluted sample.

19. The method of claim 18, wherein the eluted biomarker is isolated from the eluted glycoproteins

20. The method of 19, wherein the eluted biomarker is identified.

21. The method of claim 19, wherein the sample contains from 2 to 50 biomarkers.

22. The method of claim 18, wherein the sample is a biological fluid, a tissue preparation, or a cell culture preparation.

23. The method of claim 22, wherein the sample is a biological fluid.

24. The method of claim 23, wherein the biological fluid sample is plasma, serum, blood, urine, lacrimal secretion, seminal fluid, vaginal secretion, sweat, or cerebrospinal fluid.

25. The method of claim 18, further comprising protease treating the eluted biomarker.

26. The method of claim 25, wherein the protease is Asp-N protease, Glu-C protease, Lys-C protease, or Arg-C protease.

27. The method of claim 25, wherein the protease is trypsin.

28. The method of claim 18, further comprising cleaving the eluted biomarker with a chemical.

29. The method of claim 28, wherein the chemical is cyanogen bromide or hydroxylamine.

30. The method of claim 20, wherein the eluted biomarker is identified using mass spectroscopy.

31. The method of claim 19, wherein the biomarker is in the unbound sample removed in step (b).

32. A method of detecting a change in the glycosylation of a biomarker, the method comprising:
a. contacting the composition of claim 1 with a sample under conditions that promote binding of glycoproteins to the composition, thereby providing a bound sample;
b. washing the composition to remove unbound components of the sample, thereby forming an unbound sample;
c. eluting glycoproteins from the bound sample, thereby forming an eluted sample;
d. detecting a selected biomarker in the unbound sample or in the eluted sample; and
e. comparing the selected biomarker to a reference biomarker, wherein a difference in the biomarker in the bound or unbound sample, relative to the reference biomarker indicates a change in glycosylation of the biomarker in the sample.

33. The method of claim 32, further comprising quantitating the amount of the selected biomarker in the eluted sample or the unbound sample.

34. The method of claim 32, wherein the sample is biological sample.

35. The method of claim 34, wherein the biological sample is plasma, serum, blood, urine, lacrimal secretion, saliva, or cerebrospinal fluid.

36. The method of claim 32, wherein a selected biomarker is detected in the unbound sample.

37. A method of identifying a biomarker panel, the method comprising:
a. contacting a sample comprising at least three different lectins attached to a solid support with a sample under conditions that promote binding of glycoproteins in the sample to the lectins, thereby providing a bound sample;
b. removing unbound sample from the contacted composition;
c. eluting glycoproteins from the bound sample, thereby providing a glycoprotein sample;
d. identifying at least two proteins in the glycoprotein sample or the unbound sample, thereby providing a biomarker panel.

38. The method of claim 37, wherein the protein panel comprises at least three, four, five, ten, or fifteen glycoproteins.

39. The method of claim 37, wherein the sample is from a subject having a disease or disorder.

40. The method of claim 37, wherein the subject has or is at risk for having cancer.

41. The method of claim 37, wherein the subject has or is at risk for having breast cancer.

42. The method of claim 37, wherein the method of identifying the glycoproteins comprises the use of tandem mass spectroscopy (MS/MS), immunoassay, electrophoresis, normal phase HPLC with fluorescent detection, pulsed amperometric detection (PAD), a dye staining method, a fluorescent probe, surface plasmon resonance, MALDI-MS, MALDI-MS/MS, LC-MS/MS, LC-MS/MS, or LTQ-FTMS.

43. The method of claim 37, wherein the method of identifying the proteins comprises an enzyme-linked immunosorbent assay (ELISA), dot blot, Western blot, two-dimensional gel electrophoresis, or capillary electrophoresis.

44. The method of claim 37, further comprising constructing a diagnostic glycoprotein panel by combining glycoprotein panels from at least two subjects having related conditions.

45. The method of claim 44, wherein the subjects have been diagnosed with or at risk for having a selected disease or disorder.

46. The method of claim 44, wherein the subjects have been diagnosed with cancer.

47. The method of claim 44, wherein the subjects have been diagnosed with breast cancer.

48. The method of claim 37, wherein the subject has or is at risk for having cardiovascular disease.

49. The method of claim 37, wherein a glycan is captured.

50. The method of claim 49, wherein the glycan is a mucin or a glycosoaminoglycan.

51. A method of diagnosing the presence of a disease or disorder in a subject, the method comprising:
a. contacting a composition of claim 1 with a sample from a subject under conditions that promote binding of glycoproteins in the sample to the lectins, thereby providing a bound sample;
b. removing an unbound sample from the contacted composition;
c. eluting the glycoproteins from the bound sample;
d. identifying the presence of at least two glycoprotein biomarkers in the sample,
wherein the presence of the biomarkers indicates the presence of a disease or disorder in the subject.

52. A method of identifying a subject at risk for a disease or disorder, the method comprising:
a. contacting a composition of claim 1 with a sample from a subject suspected of being at risk for a disease of disorder under conditions that promote binding of glycoproteins in the sample to the lectins, thereby providing a bound sample;
b. removing an unbound sample from the contacted composition;
c. eluting the glycoproteins from the bound sample;
d. analyzing the sample for at least two glycoprotein biomarkers that can indicate that a subject is at risk for a disease or disorder.
